(19) United States
(12) Patent Application Publication
(10) Pub. No.: US 2025/0034092 A1
(43) Pub. Date: Jan. 30, 2025

(54) Pyridinium Compounds With Low Reduction Potentials And Persistent Radical States

(71) Applicant: Board of Trustees of Michigan State University, East Lansing, MI (US)

(72) Inventors: Thomas F. Guarr, West Olive, MI (US); David Patrick Hickey, East Lansing, MI (US)

(73) Assignee: Board of Trustees of Michigan State University, East Lansing, MI (US)

(21) Appl. No.: 18/708,156

(22) PCT Filed: Nov. 17, 2022
(86) PCT No.: PCT/US2022/050232

§ 371 Date: May 7, 2024

Related U.S. Application Data
(60) Provisional application No. 63/280,344, filed on Nov. 17, 2021.

(51) Int. Cl. C07D 213/20; C07D 213/26; C07D 213/30; C07D 213/38; H01M 8/02; H01M 8/18

(52) U.S. Cl. CPC C07D 213/20; C07D 213/26; C07D 213/30; C07D 213/38; H01M 8/02; H01M 8/188; H01M 2300/0025

(57) ABSTRACT

A series of pyridinium compounds have a low reduction potential and a highly persistent radical state are generally 2,6-dialkyl-4-arylpyridinum compounds and are utilized as anolytes in redox flow, thin-film metal-organic hybrid and all-organic batteries. Associated routes for synthesizing these pyridinium compounds from pyrylium intermediates are also disclosed.

### CROSS REFERENCE TO RELATED APPLICATIONS

This application claims priority to and the benefit of U.S. Provisional Patent Application 63/280,344, filed Nov. 17, 2021, the disclosure of which is hereby incorporated by reference in its entirety.

### FIELD OF THE DISCLOSURE

The subject application relates generally to the preparation of pyridinium compounds and their derivatives.

### BACKGROUND OF DISCLOSURE

Numerous pyridinium compounds have been explored as anolytes in redox flow batteries, but it has been difficult to achieve all of the desirable characteristics in a single system, namely a low reduction potential, high solubility, and an extremely stable radical state. Recent work (Christo S. Sevov, David P. Hickey, Monique E. Cook, Sophia G. Robinson, Shoshanna Barnett, Shelley D. Minteer, Matthew S. Sigman, and Melanie S. Sanford, Journal of the American Chemical Society 2017 139 (8), 2924-2927 DOI: 10.1021/jacs.7b00147) has shown that predictive modeling can be used to reveal important structure/property relationships, thus generating new synthetic targets. This publication noted that certain pyridinium compounds would likely possess particularly attractive properties; however, such materials proved virtually impossible to synthesize by conventional routes.

The subject application seeks to address these issues by identifying new pyridinium compounds and associated methods for forming same.

### SUMMARY OF THE DISCLOSURE

The subject application provides pyridinium compounds having low reduction potentials (−1.54 to −1.83 V vs Fc/Fc+) and highly persistent radical states. The subject application also provides associated high yield, low-cost routes for synthesis of these compounds.

In certain embodiments, the pyridinium compounds are according to formula (1A):

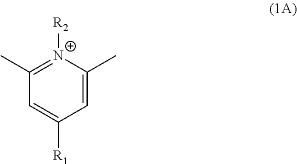

In formula (1A), R₁ is selected from substituted aryl groups and unsubstituted aryl groups and substituted and unsubstituted heteroaromatic groups.

In formula (1A), R₂ is selected from substituted and unsubstituted aryl groups, unbranched and branched alkyl, haloalkyl, aralkyl, alkyl ether, alkyl nitro, alkyl nitrile, trialkylammonium alkyl, alkyl carboxylate, alkyl sulfonate, and alkyl phosphonate groups.

In certain other embodiments, the pyridinium compounds are according to formula (1B):

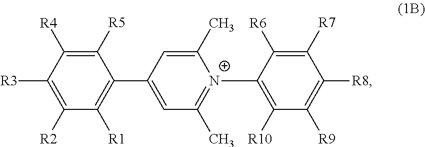

In formula (1B), R1 and R5 are independently selected from hydrogen, methyl, ethyl, halide, halomethyl, perhalomethyl, alkoxy, nitrile and nitro groups. Also, R2, R3 and R4 are independently selected from hydrogen, an unbranched alkyl, a branched alkyl, alkoxy, haloalkyl, aralkyl, alkyl ether, nitro, nitrile, halide, acetyl, trialkylammonium alkyl, alkyl carboxylate, alkyl sulfonate, and alkyl phosphonate groups. Still further, R6 and R10 are independently selected from hydrogen, methyl, ethyl, halide, halomethyl, perhalomethyl, hydroxyl, alkoxy, nitrile and nitro groups. In addition, R7 and R9 are independently selected from hydrogen, an unbranched alkyl, a branched alkyl, haloalkyl, perhalomethyl, aryl, aralkyl, alkyl ether, nitro, nitrile, halide, acetyl, trialkylammonium alkyl, alkyl carboxylate, alkyl sulfonate, alkyl phosphonate, t-Bu (tert-butyl), —CF₃, —Br, N,N-dimethylaniline, and alkyl triether groups. R8 is independently selected from hydrogen, an unbranched alkyl, a branched alkyl, alkoxy, haloalkyl, perhalomethyl, aryl, aralkyl, alkyl ether, nitro, nitrile, halide, acetyl, trialkylammonium alkyl, alkyl carboxylate, alkyl sulfonate, alkyl phosphonate groups, t-Bu (tert-butyl), —CF₃, —Br, N,N-dimethylaniline, and alkyl triether groups. In addition, at least one of R1-R10 is not hydrogen. In certain embodiments, formula (1B) also provides wherein at least one of R1, R5, R6 or R10 is not hydrogen.

In still another alternative embodiment, the pyridinium compounds are according to formula (1C):

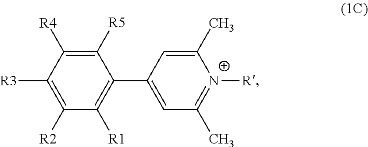

In formula (1C), R1 and R5 are independently selected from hydrogen, methyl, methoxy, ethyl, halide, halomethyl, hydroxyl, alkoxy, nitrile and nitro groups. In addition, R2, R3 and R4 are independently selected from hydrogen, an unbranched alkyl, a branched alkyl, alkoxy including methoxy, haloalkyl, aryl, aralkyl, alkyl ether, nitro, nitrile, halide, acetyl, trialkylammonium alkyl, alkyl carboxylate, alkyl sulfonate, and alkyl phosphonates groups. Still further, R′ is selected from unbranched and branched alkyl, haloalkyl, aralkyl, alkyl ether, alkyl nitro, alkyl nitrile, trialkylammonium alkyl, alkyl carboxylates, alkyl sulfonates, and alkyl phosphonate groups. In these embodiments, any one or more of R1, R2, R3, R4 and R5 may be the methoxy group when R′ has 2 carbons or more. In certain embodiments, formula (1C) also provides wherein at least one of R1 or R5 is not hydrogen.

In certain further embodiments, the pyridinium compounds, which may or may not encompass one or more of formulas (1A) (1B), or (1C), are selected from pyridinium compounds (1D)-(1Z) and (1AA)-(1LL) below as follows:

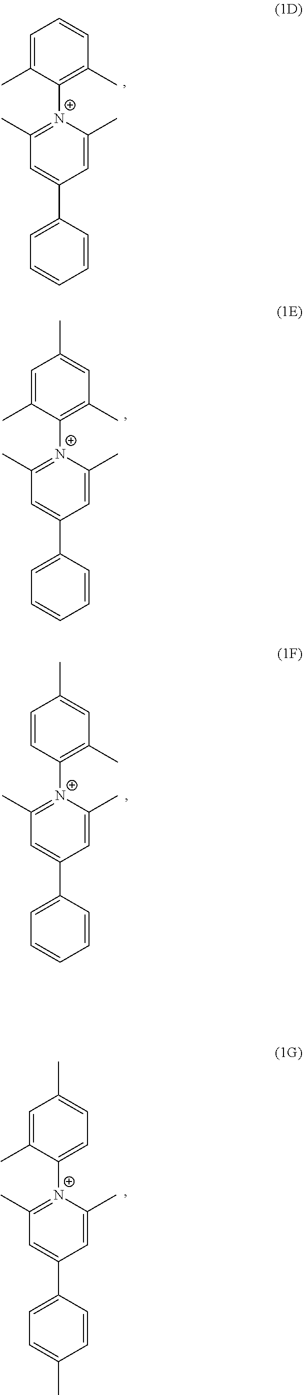

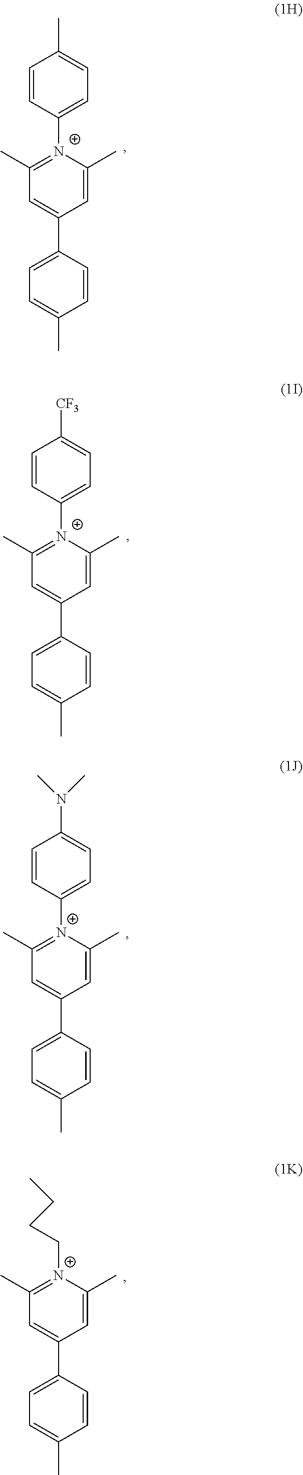

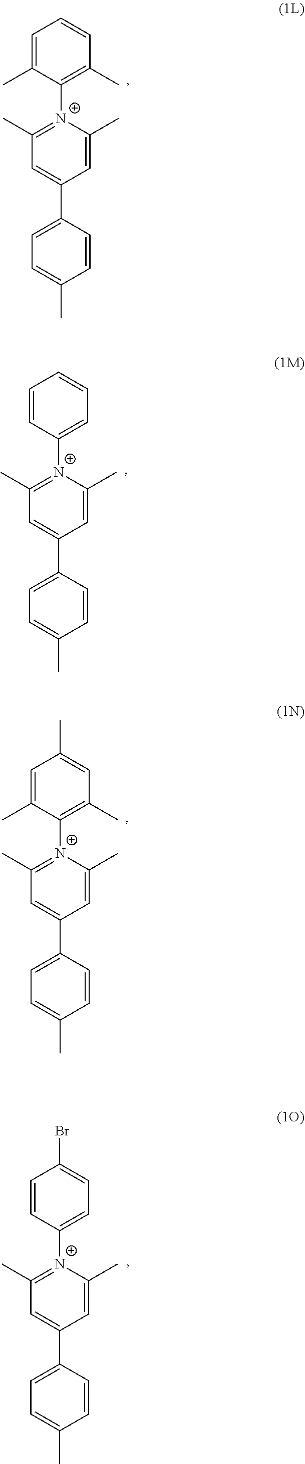

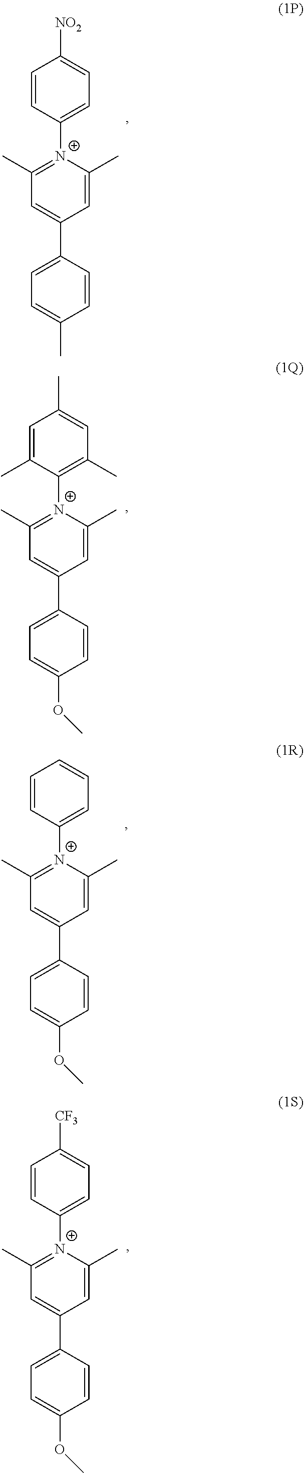

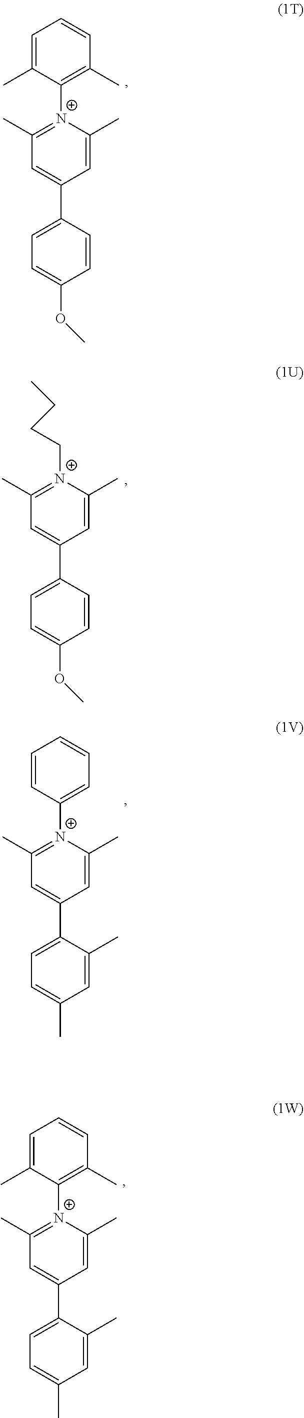

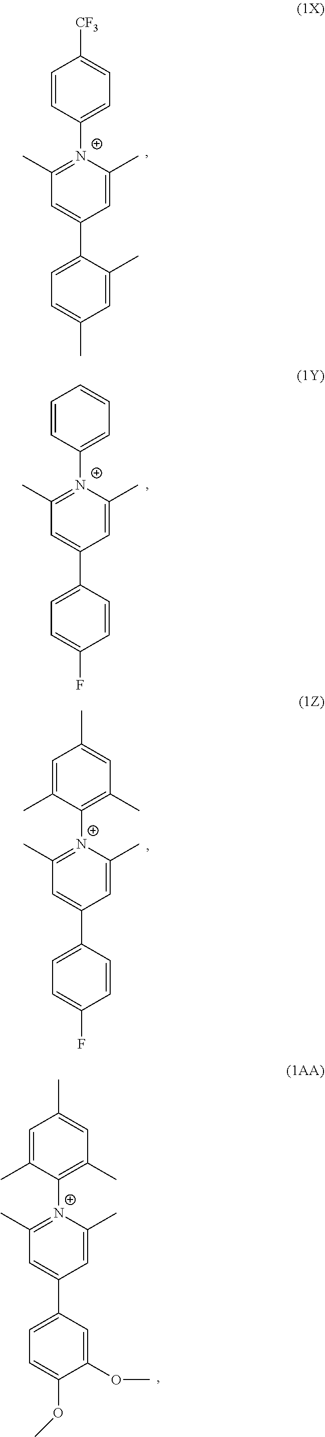

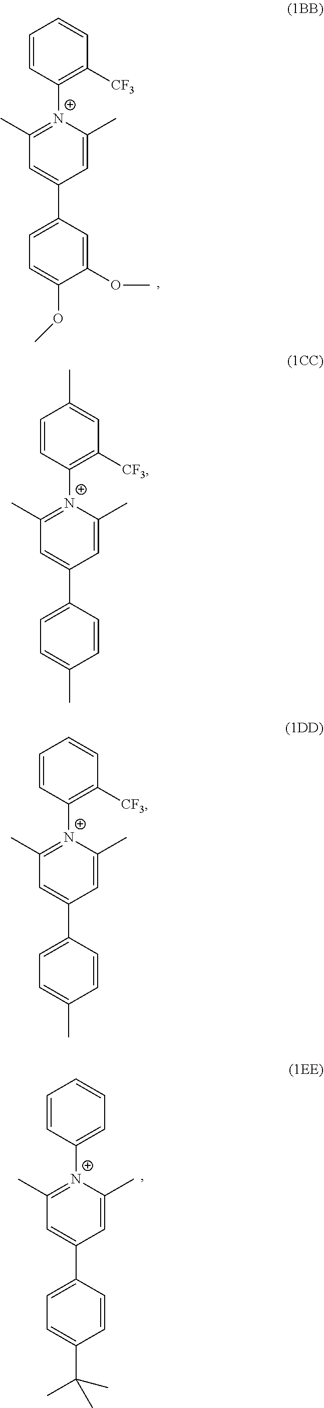

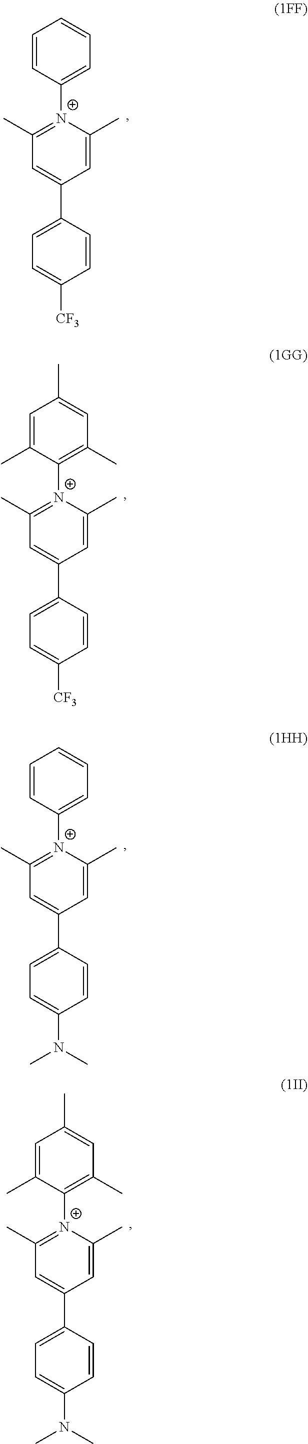

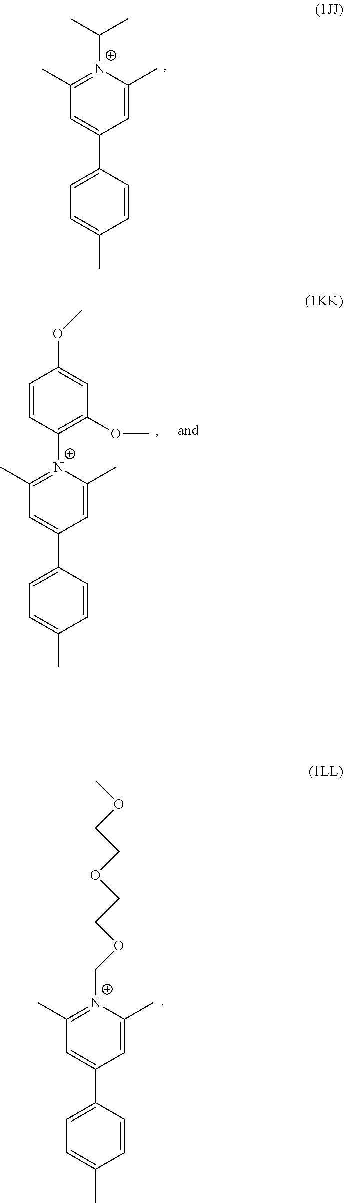

In particular, the subject application provides an extremely wide variety of 2,6-dialkyl-4-arylpyridinum compounds according to any one or more of formulas (1A)-(1Z) and (1AA)-(1LL) are accessible via pyrylium intermediates.

Several of these pyridinium compounds corresponding to any one or more of formulas (1A)-(1Z) and (1AA)-(1LL) are found to display excellent solubility in polar organic solvents. Further, in certain embodiments, the introduction of a simple methyl group at R3 according to formula (1B) to form the 2,6-dialkyl-4-tolylpyridinum compound according to the subject application increased the solubility by more than 3 times as compared with the pyridinium compound not including a methyl group at R3 but otherwise including the same other substituents, which suggests that very high energy density nonaqueous flow batteries may be within reach. Further, these pyridinium compounds according to any one or more of formulas (1A)-(1Z) and (1AA)-(1LL) have exceptionally high diffusion coefficients (typically on the order of 1.1 to 1.5×10⁻⁵ cm²/s), which may promote high power densities.

The 2,6-dialkyl-4-arylpyridinum compounds according to any one or more of formulas (1A)-(1Z) and (1AA)-(1LL) of the subject application undergo chemically and electrochemically reversible one-electron reduction at low potentials (ca. (about) −1.54 to −1.83 V vs. Fc/Fc+,). Electrode kinetics are favorable in all cases, as illustrated in representative cyclic voltammogram of a 1 mM pyridinium salt described in FIG. 2 below. Moreover, the compounds are extremely soluble in polar organic solvents (over 1M in acetonitrile for several derivatives) and display remarkably high diffusion coefficients.

Moreover, the synthesis of compounds according to Formula (1B), when R6 and/or R10 is not hydrogen, was surprising and unexpected due to steric crowding. Likewise, the formation of persistent radical states in compounds according to Formula (1B) where R1 and/or R5 is not hydrogen is also surprising, given the fact that π overlap is substantially reduced by restricted rotation around the pyridinium-(4-aryl) bond.

The subject application also discloses a redox flow batteries which include a charge-carrying electrolyte including the pyridinium compounds according to any one or more of formulas (1A)-(1Z) and (1AA)-(1LL) as provided herein.

Other features and advantages of the subject application will be readily appreciated, as the same becomes better understood, after reading the subsequent description taken in conjunction with the accompanying drawings.

### BRIEF DESCRIPTION OF THE DRAWINGS

The advantages of the subject application will be readily appreciated as the same becomes better understood by reference to the following detailed description when considered in connection with the accompanying drawing. It is to be understood that the drawings are purely illustrative and not necessarily drawn to scale.

FIG. 1 is a schematic illustration of redox flow battery according to one non-limiting embodiment of the subject application.

FIG. 2 is an illustration of a cyclic voltammogram of a representative pyridinium compound (1 mM pyridinium salt) in accordance with the subject application.

### DESCRIPTION OF THE PREFERRED EMBODIMENT(S)

The subject application provides pyridinium compounds having desired low reduction potentials (i.e., from about −1.54 to −1.83 V vs Fc/Fc⁺, such as from 1.54 to −1.83 V vs Fc/Fc⁺) and highly persistent radical states and associated high yield, low-cost route to synthesis of these compounds.

As used herein, the term “ca.” or “about”, as it relates to reduction potentials or percentage yields, allows for a variability of a number or range of numbers as described herein of 5%.

Still further, the description of an “R” group in any of the representative formulas may be described below with or without the term group but are intended to be equivalent. By way of one non-limiting example, in a description below where a particular R group is being described as “ . . . selected from methyl”, it is understood that the term “methyl” can be used interchangeably with the phrase “methyl group” and is meant to refer to a CH₃ group that is positioned in the position located by R in the respective formula. The same holds true for any groups described in any one or more of formulas (1A)-(1Z) and (1AA)-(1LL) described below.

The pyridinium compounds are ideally suited for use as anolytes in redox flow batteries owing to the afore-mentioned low reduction potentials, high solubility, and highly persistent radical states.

In certain embodiments, the pyridinium compounds are according to formula (1A):

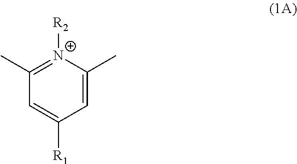

In formula (1A), R₁ is selected from substituted aryl groups, unsubstituted aryl groups, and substituted and unsubstituted heteroaromatic groups. More specific R₁ groups that may be utilized include aryl or substituted aryl groups such as phenyl, 4-tolyl, 4-fluorophenyl, 4-methoxyphenyl, 3,4-dimethoxyphenyl, 2,4-dimethylphenyl, 4-dimethylaminophenyl, 4-(t-Bu)phenyl, 4-trifluoromethylphenyl, and 4-(alkoxyether)phenyl groups.

In formula (1A), R₂ is selected from substituted and unsubstituted aryl groups, unbranched and branched alkyl, haloalkyl, aralkyl, alkyl ether, alkyl nitro, alkyl nitrile, trialkylammonium alkyl, alkyl carboxylate, alkyl sulfonate, and alkyl phosphonate groups.

In certain further embodiments, the pyridinium compounds are according to formula (1B):

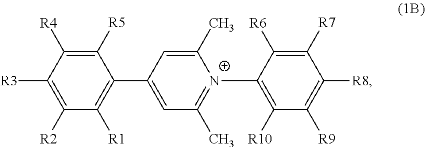

In formula (1B), R1 and R5 are independently selected from hydrogen, methyl, ethyl, halide, halomethyl, perhalomethyl, alkoxy, nitrile and nitro groups. Also, R2, R3 and R4 are independently selected from hydrogen, an unbranched alkyl, a branched alkyl, alkoxy, haloalkyl, aralkyl, alkyl ether, nitro, nitrile, halide, acetyl, trialkylammonium alkyl, alkyl carboxylate, alkyl sulfonate, and alkyl phosphonate groups. Still further, R6 and R10 are independently selected from hydrogen, methyl, ethyl, halide, halomethyl, perhalomethyl, hydroxyl, alkoxy, nitrile and nitro groups. In addition, R7 and R9 are independently selected from hydrogen, an unbranched alkyl, a branched alkyl, haloalkyl, perhalomethyl, aryl, aralkyl, alkyl ether, nitro, nitrile, halide, acetyl, trialkylammonium alkyl, alkyl carboxylate, alkyl sulfonate, alkyl phosphonate group, t-Bu (tert-butyl), —CF₃, —Br, N,N-dimethylaniline, and alkyl triether groups. R8 is independently selected from hydrogen, an unbranched alkyl, a branched alkyl, alkoxy, haloalkyl, perhalomethyl, aryl, aralkyl, alkyl ether, nitro, nitrile, halide, acetyl, trialkylammonium alkyl, alkyl carboxylate, alkyl sulfonate, and alkyl phosphonate groups, t-Bu (tert-butyl), —CF₃, —Br, N,N-dimethylaniline, and alkyl triether. In addition, at least one of R1-R10 is not hydrogen.

In certain embodiments, formula (1B) also provides wherein at least one of R1, R5, R6 or R10 is not hydrogen.

In still further embodiments, the pyridinium compounds are according to formula (1C):

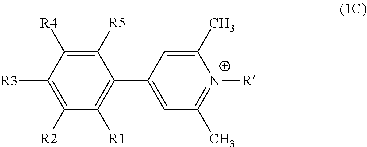

In formula (1C), R1 and R5 are independently selected from hydrogen, methyl, methoxy, ethyl, halide, halomethyl, hydroxyl, alkoxy such as methoxy, nitrile and nitro groups.

In addition, R2, R3 and R4 are independently selected from hydrogen, an unbranched alkyl, a branched alkyl, alkoxy, methoxy, haloalkyl, aryl, aralkyl, alkyl ether, nitro, nitrile, halide, acetyl, trialkylammonium alkyl, alkyl carboxylate, alkyl sulfonate, and alkyl phosphonate groups. Still further, R′ is selected from unbranched and branched alkyl, haloalkyl, aralkyl, alkyl ether, alkyl nitro, alkyl nitrile, trialkylammonium alkyl, alkyl carboxylates, alkyl sulfonates, and alkyl phosphonate groups. In these embodiments, any one or more of R1, R2, R3, R4 and R5 may be the methoxy group when R′ has 2 carbons or more.

In certain embodiments, formula (1C) also provides wherein at least one of R1 or R5 is not hydrogen.

In certain further embodiments, the pyridinium compounds, which may or may not encompass one or more of formulas (1A) (1B), or (1C), are selected from pyridinium compounds (1D)-(1Z) and (1AA)-(1LL) below as follows:

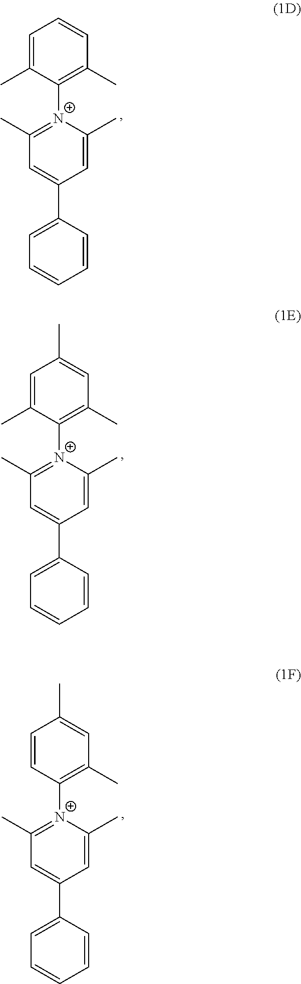

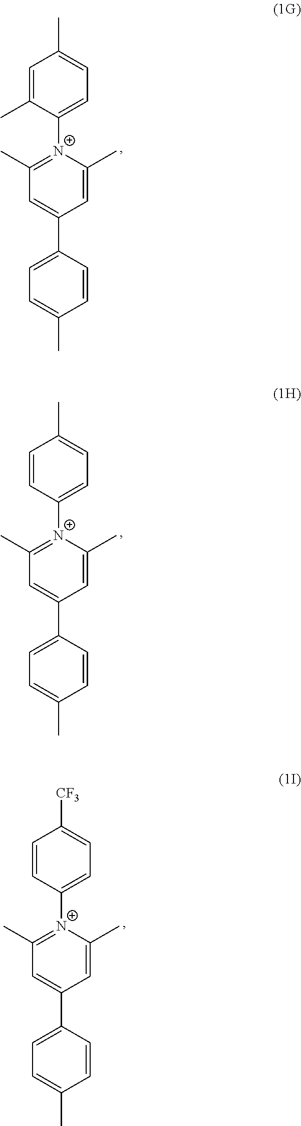

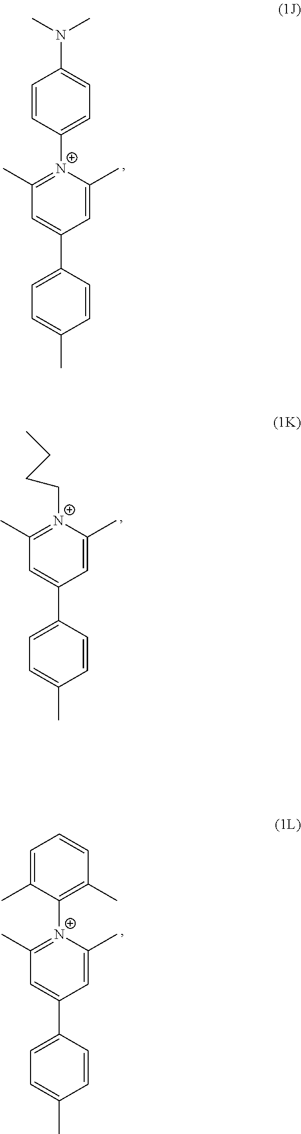

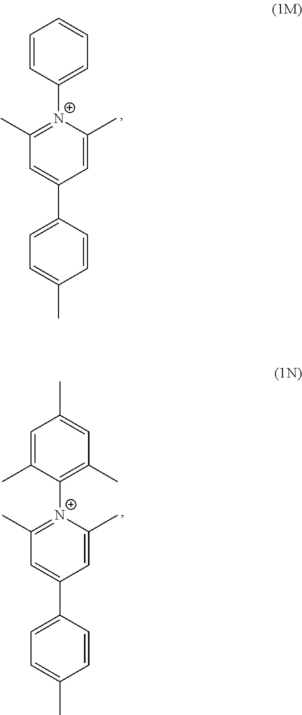

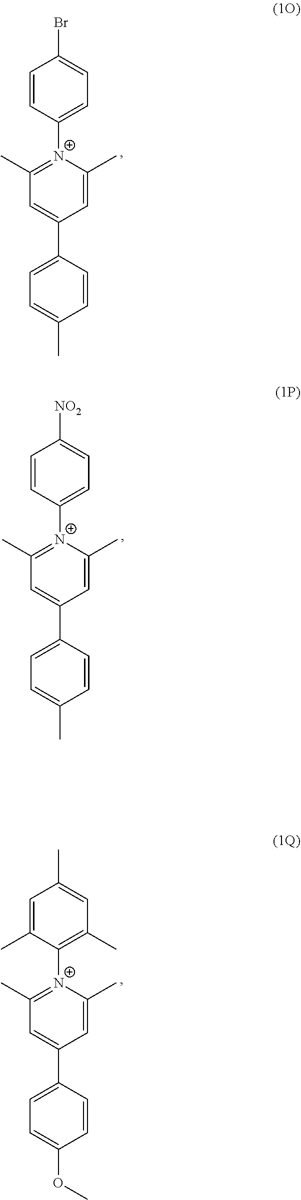

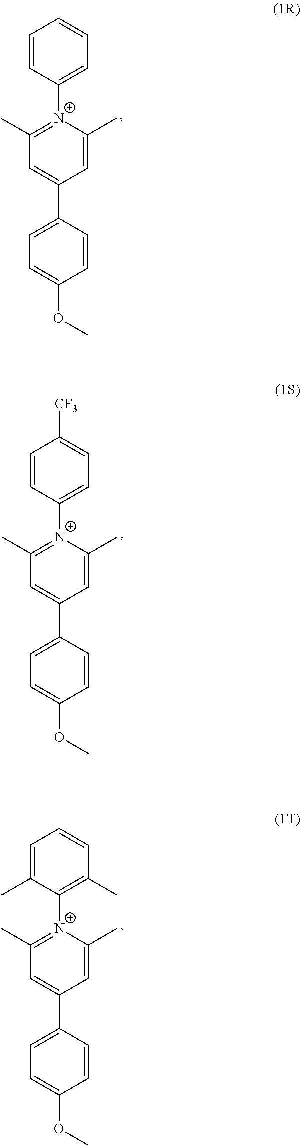

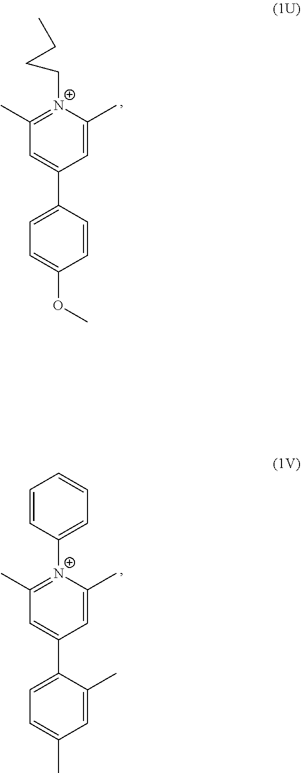

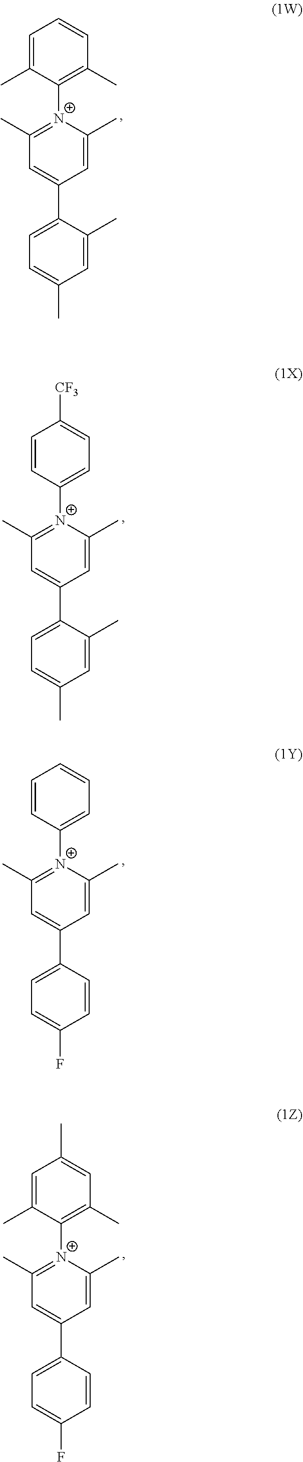

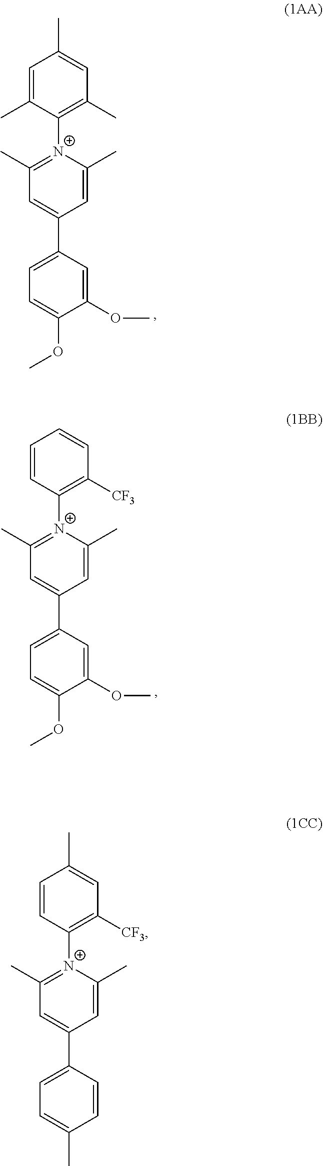

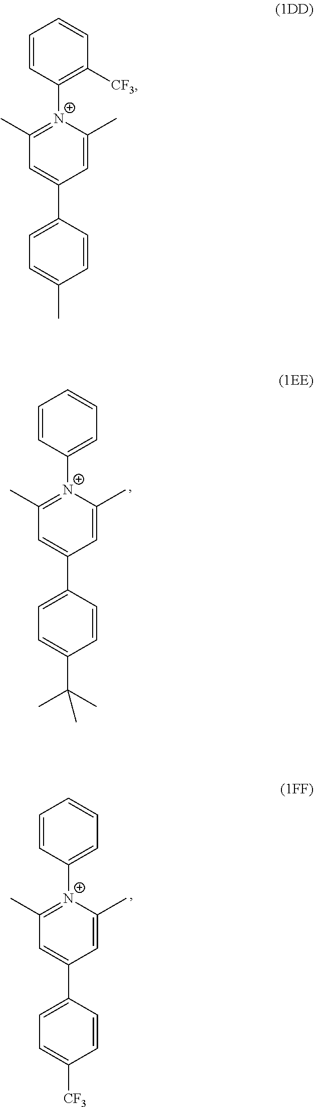

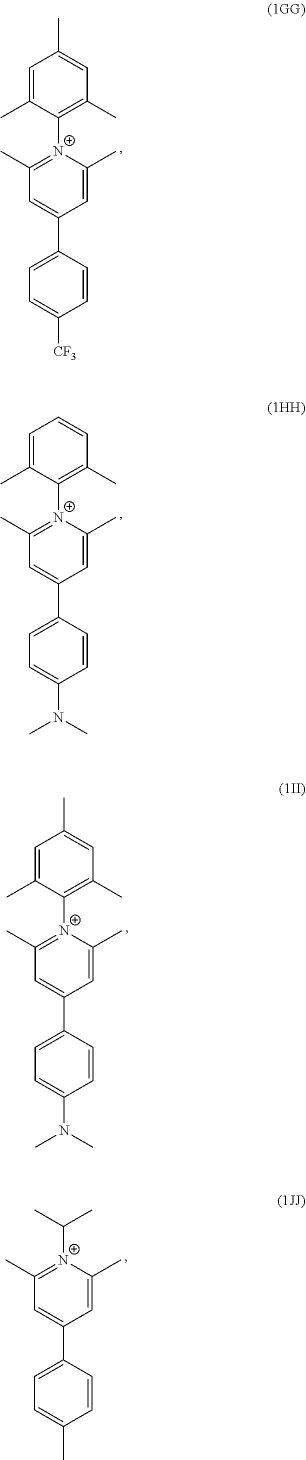

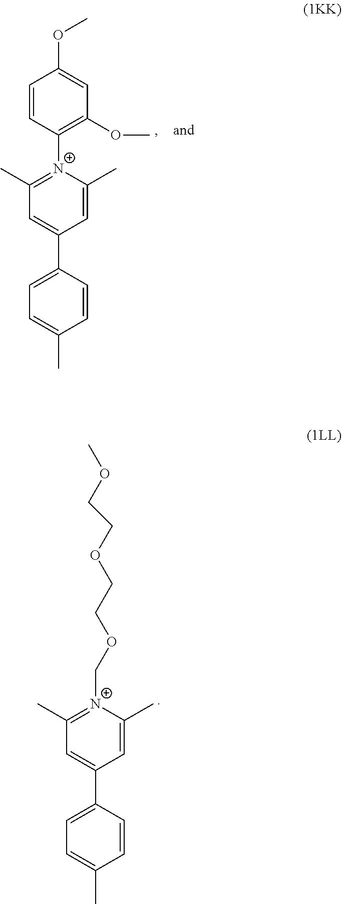

The subject application also discloses synthesis routes for forming the pyridinium compounds according to formulas (1A), (1B) and (1C) and the other individual pyridinium compounds according to formulas (1D)-(1Z) and (1AA)-(1LL) described above.

In general, a wide variety of 2,6-dialkyl-4-arylpyridinum compounds according to formula (1A) (and in certain instances formulas (1B) and (1C) and the other individual pyridinium compounds according to formulas (1D)-(1Z) and (1AA)-(1LL) described above) are accessible via pyrylium intermediates (III) and can be formed as represented according to Reaction Scheme (A) below.

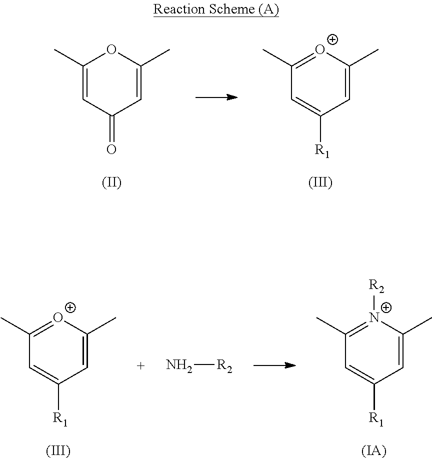

Possible N-substituents (i.e., R₂ substituents) for formula (1A) provided via NH₂—R₂ in Reaction Scheme (A) include essentially any group that is compatible with a terminal amine, including alkyl, aralkyl, alkyl ether, haloalkyl, trialkylammonium alkyl, aryl, substituted aryl, among other N-substituents.

Exemplary R₁ groups include phenyl, tolyl, methoxyphenyl, xylyl, mesityl, perfluorophenyl, and trifluoromethylphenyl groups.

Exemplary embodiments of the subject application according to formula (1B) (and in certain instances formula (1C)) may be formed in accordance with Reaction Scheme (B) and Table (1) as follows:

Reaction Scheme (B) and Table 1

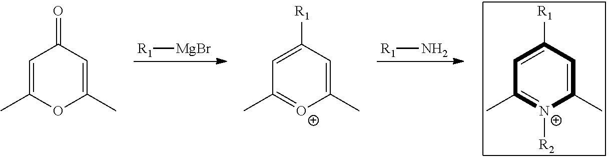

| | | E_{1/2} | solu- | solu- | solu- | |
|---|---|---|---|---|---|---|
| | | V vs | bility | bility | bility | |
| | | Fc/ | mol/L | mol/L | mol/L | |
| R₁ | R₂ | Fc⁺ | in ACN | in PC | in THF | parent pyrylium |
| 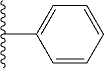 [SMILES: *c1ccccc1] | 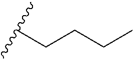 [SMILES: *CCCC] | −1.70 | 0.406 ± 0.007 | 0.64 ± 0.01 | | 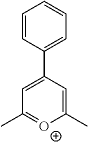 |
| | 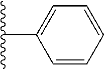 [SMILES: *c1ccccc1] | −1.59 | 0.55 ± 0.02 | 0.618 ± 0.005 | 0.0011 ± 0.0002 | |
| | 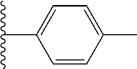 [SMILES: *c1ccc(C)cc1] | −1.61 | 0.32 ± 0.03 | | | |
| | 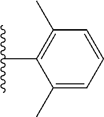 [SMILES: *c1c(C)cccc1C] | −1.59 | 0.302 ± 0.007 | | 0.00040 ± 0.00002 | |
| | 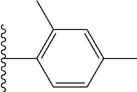 [SMILES: *c1ccc(C)cc1C] | −1.60 | 0.99 ± 0.02 | | 0.0039 ± 0.0002 | |
| | 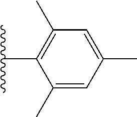 [SMILES: *c1c(C)cc(C)cc1C] | −1.60 | 0.360 ± 0.002 | | | |
| | 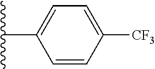 [SMILES: *c1ccc(C(F)(F)F)cc1] | −1.54 | 1.2 ± 0.1 | | | |
| | 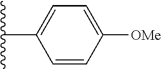 [SMILES: *c1ccc(OC)cc1] | | 0.55 ± 0.05 | | 0.00160 ± 0.00007 | |
| | 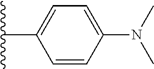 [SMILES: *c1ccc(N(C)C)cc1] | | | | | |
| | 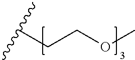 | | | | | |
| 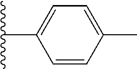 [SMILES: *c1ccc(C)cc1] | 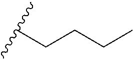 [SMILES: *CCCC] | −1.73 | 1.40 ± 0.04 | 0.884 ± 0.008 | | 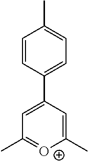 |
| | 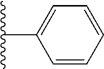 [SMILES: *c1ccccc1] | −1.63 | 0.45 ± 0.03 | | 0.0010 ± 0.0002 | |
| | 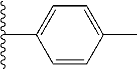 [SMILES: *c1ccc(C)cc1] | −1.64 | 1.06 ± 0.03 | | 0.0071 ± 0.0002 | |
| | 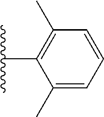 [SMILES: *c1c(C)cccc1C] | −1.63 | 0.75 ± 0.02 | | | |
| | 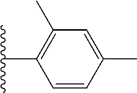 [SMILES: *c1ccc(C)cc1C] | −1.64 | 1.00 ± 0.02 | | 0.0111 ± 0.0004 | |
| | 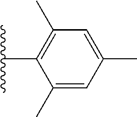 [SMILES: *c1c(C)cc(C)cc1C] | −1.64 | 0.422 ± 0.007 | | | |
| | 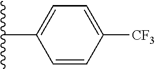 [SMILES: *c1ccc(C(F)(F)F)cc1] | −1.58 | 0.56 ± 0.05 | | | |
| | 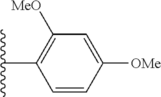 [SMILES: *c1ccc(OC)cc1OC] | | 1.3 ± 0.1 | | | |
| | 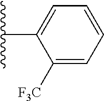 [SMILES: *c1ccccc1C(F)(F)F] | | | | | |
| | 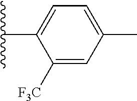 [SMILES: *c1ccc(C)cc1C(F)(F)F] | | | | | |
| | 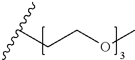 | −1.71 | 1.20 ± 0.03 | | | |
| | 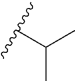 [SMILES: *C(C)C] | | | | | |
| 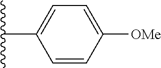 [SMILES: *c1ccc(OC)cc1] | 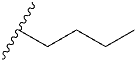 [SMILES: *CCCC] | −1.78 | 1.13 ± 0.04 | | | 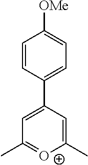 |
| | 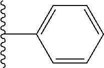 [SMILES: *c1ccccc1] | −1.68 | 0.55 ± 0.05 | | | |
| | 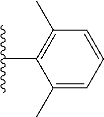 [SMILES: *c1c(C)cccc1C] | −1.67 | 10.1 ± 0.09 | | | |
| | 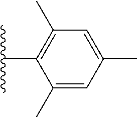 [SMILES: *c1c(C)cc(C)cc1C] | −1.68 | 2.1 ± 0.2 | | 0.0037 ± 0.0002 | |
| | 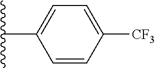 [SMILES: *c1ccc(C(F)(F)F)cc1] | −1.62 | 1.01 ± 0.07 | | | |
| 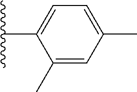 [SMILES: *c1ccc(C)cc1C] | 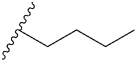 [SMILES: *CCCC] | −1.82 | | | | 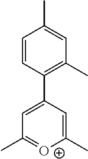 |
| | 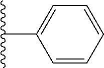 [SMILES: *c1ccccc1] | −1.72 | 0.97 ± 0.04 | | | |
| | 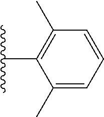 [SMILES: *c1c(C)cccc1C] | −1.71 | 1.08 ± 0.01 | | 0.00210 ± 0.00004 | |
| | 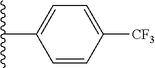 [SMILES: *c1ccc(C(F)(F)F)cc1] | −1.65 | 0.43 ± 0.09 | | | |
| 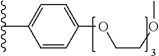 | 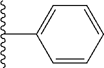 [SMILES: *c1ccccc1] | | | | | 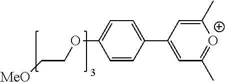 |
| 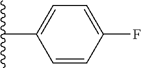 [SMILES: *c1ccc(F)cc1] | 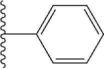 [SMILES: *c1ccccc1] | | | | | 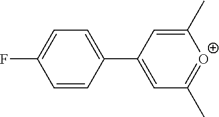 |
| | 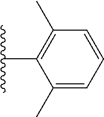 [SMILES: *c1c(C)cccc1C] | | | | | |
| 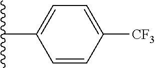 [SMILES: *c1ccc(C(F)(F)F)cc1] | 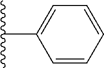 [SMILES: *c1ccccc1] | | | | | 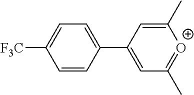 [SMILES: Cc1cc(-c2ccc(C(F)(F)F)cc2)cc(C)[o+]1] |
| | 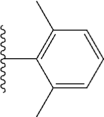 [SMILES: *c1c(C)cccc1C] | | | | | |
| 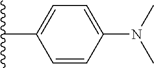 [SMILES: *c1ccc(N(C)C)cc1] | 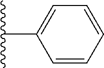 [SMILES: *c1ccccc1] | | | | | 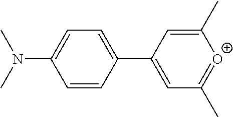 |
| | 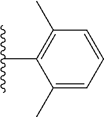 [SMILES: *c1c(C)cccc1C] | | | | | |
| 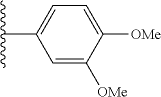 [SMILES: *c1ccc(OC)c(OC)c1] | 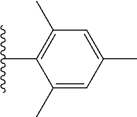 [SMILES: *c1c(C)cc(C)cc1C] | | | | | 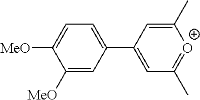 [SMILES: COc1ccc(-c2cc(C)[o+]c(C)c2)cc1OC] |
| | 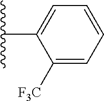 [SMILES: *c1ccccc1C(F)(F)F] | | | | | |
| 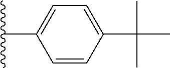 [SMILES: *c1ccc(C(C)(C)C)cc1] | 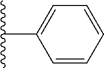 [SMILES: *c1ccccc1] | | | | | 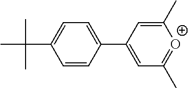 [SMILES: Cc1cc(-c2ccc(C(C)(C)C)cc2)cc(C)[o+]1] |

In Reaction Scheme (B) shown above, dimethyl-γ-pyrone is reacted with an R₁-magnesium bromide (such as phenylmagnesium bromide or methoxy-phenylmagnesium bromide as shown in Table 1), to form a pyrylium intermediate. The pyrylium intermediate is then reacted with an amine-containing compound, represented by NH₂—R₂, to yield the pyridinium compounds according to formula (1B).

Possible N-substituents (i.e., R₂ substituents) provided via NH₂—R₂ in Reaction Scheme (B) include essentially any group that is compatible with a terminal amine, including alkyl, aralkyl, alkyl ether, haloalkyl, trialkylammonium alkyl, aryl, substituted aryl, among other N-substituents and including each of the specific groups listed in Table 1 above.

As noted above, the pyridinium compounds in accordance with the subject application (including formulas (1A), (1B) and (1C) and the other individual pyridinium compounds according to formulas (1D)-(1Z) and (1AA)-(1LL) described above are suitable for use as an anolyte in a redox flow battery.

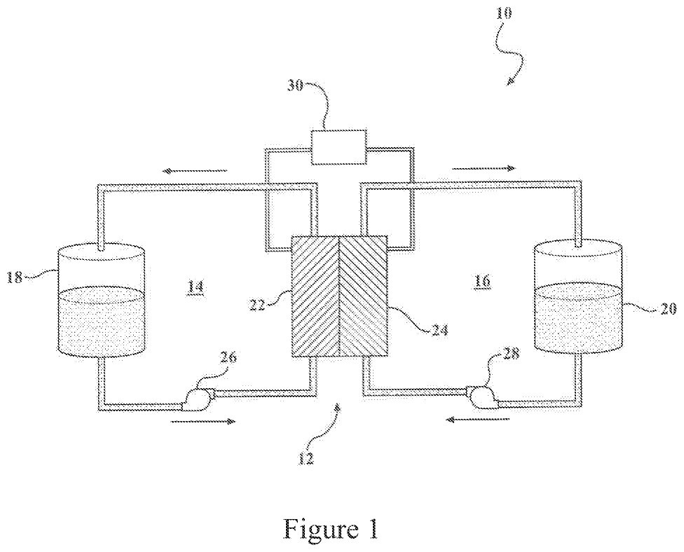

FIG. 1 illustrates a redox flow battery 10 according to an embodiment of the subject application. The redox flow battery 10 may be described as an energy storage device that utilizes redox (reduction and oxidation) reactions to generate energy, which is stored in electrolyte solutions flowing through the battery 10. During discharge of the battery 10, electrons are released during an oxidation reaction on the negative (or anode) side of the battery 10. The electrons move through an external circuit to do useful work and are thereafter accepted during a reduction reaction at the positive (or cathode) side of the battery 10. The direction of the current and the chemical reactions are reversed during charging. The energy produced by the redox flow battery 10 is often used for grid storage applications. It is to be appreciated, however, that the redox flow battery 10 can be scaled to fit the needs of any suitable application.

Turning to FIG. 1, the redox flow battery 10 has an electrochemical cell 12 including a positive (cathode) side 14 and a negative (anode) side 16. The positive side 14 has a first receptacle 18 containing a charge carrying electrolyte and the oxidized form of an electroactive material (herein referred to as an active material), and the negative side 16 has a second receptacle 20 containing the charge carrying electrolyte and the reduced form of an electroactive material. The positive side 14 further includes a cathode 22, and the negative side further has an anode 24. The net charge in each receptacle is zero. Any positively charged species are balanced by a negatively charged species, and vice versa.

During charging and discharging of the redox flow battery 10, the charge-carrying electrolyte on the positive side 14 circulates from the first receptacle 18 and through the cathode 22 by a first pump 26. The charge-carrying electrolyte on the negative side 16 circulates from the second receptacle 20 and through the anode 24 by a second pump 28. The cathode 22 and the anode 24 may be electrically connected through current collectors with an external load 30. As the electrolyte pass through the cathode 22 and the anode 24, the electroactive material reacts (via redox reaction(s)) to generate energy.

The cathode 22 may be one or a pair of electrodes or an array of electrodes. The anode 24 may be one or a pair of electrodes or an array of electrodes. The cathode 22 and the anode 24 are not particularly limited and may be any known in the art. In a non-limiting example, one or more of the cathode 22 and the anode 24 is a carbon-based electrode, a metal-based electrode, and combinations thereof. Non-limiting examples of carbon-based electrodes include electrodes made or formed from porous carbon (e.g., carbon felt, carbon paper and graphite felt), carbon nanotubes, carbon nanowires, graphene, and/or the like, and/or combinations thereof. Non-limiting examples of metal-based electrodes include electrodes made or formed from gold, steel, nickel, platinum-coated gold, platinum-coated carbon, and/or the like, and/or combinations thereof. In another non-limiting example, the cathode 22 and/or the anode 24 is porous. The cathode 22 and/or anode 24 may further include additives, such as carbon black, flake graphite, and/or the like. Each of the cathode 22 and the anode 24 may be in any convenient form, including foils, plates, rods, screens, pastes, or as a composite made by forming a coating of the electrode material on a conductive current collector or other suitable support.

The charge-carrying electrolyte includes a charge-carrying medium (i.e., a solvent or gel, an electrolyte salt and one or more redox-active materials (i.e., the pyridinium compounds according to any one of Formulas (1A), (1B). (1C), (1D)-(1Z) or (1AA)-(1LL) described above)) and ions. The charge-carrying medium may be one or more liquids and/or gels. In addition, the charge-carrying medium may be used over a wide temperature range, for example, from about −30° C. to about 70° C. without freezing or boiling and is typically stable in the electrochemical window within which the cathode 22 and the anode 24 operate.

The charge-carrying medium, in certain embodiments, is present in an amount of from 10% to 100% by weight, such as from 40% to 99% by weight, such as from 60 to 99% by weight, such as from 65% to 95% by weight, or such as from 70% to 90% by weight, each based on a total weight of the charge-carrying electrolyte. All values and ranges of values within those values described above are hereby expressly contemplated in various non-limiting embodiments.

While the pyridinium compound are suitable for use in redox flow batteries, as described above, potential application of these materials may extend beyond their use in redox flow batteries. Possible additional applications may include, but are not limited to, as redox catalysts, chemical reductants (from the reduced form), as additives for conventional batteries, or in molecular electronics.

In certain exemplary embodiments, the pyridinium compound according to any one of Formulas (1A), (1B). (1C), (1D)-(1Z) or (1AA)-(1LL) described above can also be linked to an oxidizable moiety for subsequent used in the systems described above. Exemplary oxidizable moieties include but are not limited to ferrocene, carbazole, phenazine, phenoxazine, phenothiazine, phenothiazine-5-oxide, phenothiazine-5,5-dioxide, benzoquinone, TEMPO, and/or cyclopropenium.

In another embodiment, the redox-active pyridinium compounds described herein may be attached to or be a component of a polymer backbone. Such redox active polymers can then be employed as the soluble anolyte in a redox flow battery, where their large size and high charge serves to decrease crossover.

In yet another embodiment, any one of pyridinium compounds according to Formulas (1A), (1B). (1C), (1D)-(1Z) or (1AA)-(1LL) described above can also be used in metal-organic hybrid or all-organic thin film (i.e., “jelly-roll”) batteries. Such batteries take advantage of established low-cost manufacturing methods such as roll-to-roll processing and additive methods but utilize redox-active organic compounds or polymers as the active material at one or both electrodes. In these batteries, the redox-active pyridinium compounds may be attached to or be a component of a polymer backbone. Alternatively, the pyridinium compounds according to Formulas (1A), (1B). (1C), (1D)-(1Z) or (1AA)-(1LL) described above or polymers incorporating them may be covalently attached to an electrode such as a metal or carbon, carbon surface. In a non-limiting example, the electrode is a carbon-based electrode, a metal-based electrode, and combinations thereof. Non-limiting examples of carbon-based electrodes include electrodes made or formed from porous carbon (e.g., carbon felt, carbon paper and graphite felt), carbon nanotubes, carbon nanowires, graphene, and/or the like, and/or combinations thereof. Non-limiting examples of metal-based electrodes include electrodes made or formed from gold, steel, nickel, platinum-coated gold, platinum-coated carbon, and/or the like, and/or combinations thereof. The electrode may further include additives, such as carbon black, flake graphite, graphene, and/or the like. The electrode may be in any convenient form, including foils, plates, rods, screens, pastes, or as a composite made by forming a coating of the electrode material on a conductive current collector or other suitable support. Such batteries also contain a charge-carrying electrolyte. The charge-carrying electrolyte includes a charge-carrying medium (i.e., a solvent or gel, and an electrolyte salt). It may also include other electroactive materials, stabilizing agents, or other additives to improve battery performance and/or service life. The charge-carrying medium may be one or more liquids and/or gels. In addition, the charge-carrying medium may be used over a wide temperature range, for example, from about −30° C. to about 70° C. without freezing or boiling and is typically stable in the electrochemical window within which the battery operates.

See, for example, “High-Power-Density Organic Radical Batteries”, C. Friebe and U.S. Schubert, Top Curr Chem (Z) (2017) 375:19, “Sustainable Energy Storage: Recent Trends and Developments toward Fully Organic Batteries”, C. Friebe, A. Lex-Balducci, and U.S. Schubert, ChemSusChem (2019), 12, 4093-4115, or “Organic Batteries Based on Just Redox Polymers”, N. Goujon, N. Casada, N. Patil, R. Marcilla, and D. Mecerreyes, Prog. Polymer Sci (2021), 122, 101449.

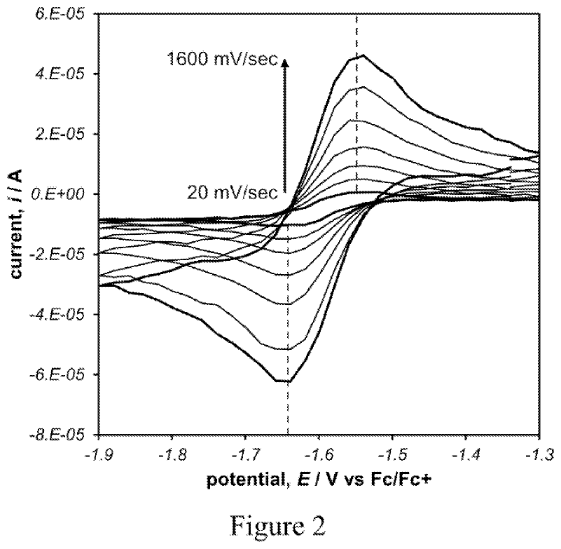

The 2,6-dialkyl-4-arylpyridinum compounds according to any one or more of formulas (1A)-(1Z) and (1AA)-(1LL) of the subject application undergo chemically and electrochemically reversible one-electron reduction at low potentials (ca. (about) −1.54 to −1.83 V vs. Fc/Fc⁺,). Electrode kinetics are favorable in all cases, as illustrated in representative cyclic voltammogram of 1 mM pyridinium salt in FIG. 2. Referring to FIG. 2, a cyclic voltammogram of 1 mM pyridinium salt was performed at variable scan rates using 100 mM Bu₄NBF₄ as a supporting electrolyte in acetonitrile with a 3 mm diameter glassy carbon electrode under 22° C. and a pure N₂ atmosphere. The invariance of peak oxidation potential and peak reduction potential at different scan rates illustrated in FIG. 2 indicates that the corresponding heterogeneous electron transfer rate constant, k^{o}, is greater than 1 cm/sec, with this value being sufficiently fast to be suitable for use in a redox flow battery.

Moreover, the pyridinium compounds of the subject application are extremely soluble in polar organic solvents (over 1M in acetonitrile for several derivatives) and display remarkably high diffusion coefficients.

Moreover, the synthesis of compounds according to Formula (1B), when R6 and/or R10 is not hydrogen, was surprising and unexpected due to steric crowding. Likewise, the formation of persistent radical states in compounds according to Formula (1B) where R1 and/or R5 is not hydrogen is also surprising, given the fact that π overlap is substantially reduced by restricted rotation around the pyridinium-(4-aryl) bond.

### Examples

### Synthesis of 2,6-dimethyl-4-phenyl pyrylium tetrafluoroborate (1a) and Derivatives (1b, 1c, and 1d

2,6-dimethyl-4-phenyl pyrylium tetrafluoroborate (1a) was synthesized by an adaptation of the procedure reported by DiMauro and Kozlowski as illustrated in Reaction Scheme (C) below (DiMauro, E. F. and Kozlowski, M. C., Phosphabenzenes as electron withdrawing phosphine ligands in catalysis. J. Chem. Soc., Perkin Trans. 1 2002, 439-444). An oven dried 250 milliliter round bottom flask was charged with a magnetic stir bar and 2,6-dimethyl-y-pyrone (2.0 grams, 0.016 moles) was dissolved in tetrahydrofuran (THF, 75 milliliters) while under nitrogen. Phenylmagnesium bromide as a 1 molar solution in THF (16 milliliters, 0.016 moles) was added to an addition funnel via a cannula. After cooling the solution to five degrees Celsius (5° C.) via an ice bath, the contents of the addition funnel were added dropwise to the solution. The resultant crude solution was allowed to warm to room temperature over an hour. The crude solution was then poured over boron trifluoride diethyl etherate (BF₄⁻, 5.92 milliliters, 0.048 mole) to yield a yellow precipitate which was filtered and washed with diethyl ether. After recrystallizing in methanol, the product according to formula (1a) shown below was isolated as a yellow solid (3.0157 g, 0.011 mol) in 69% yield.

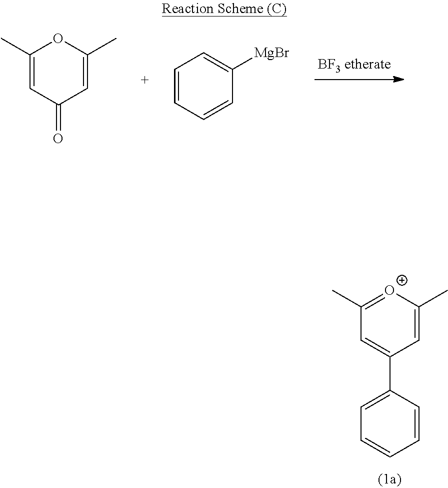

Derivatives (1b-j) shown below were synthesized following the above procedure of Reaction Scheme (C) above with the corresponding Grignard reagent:

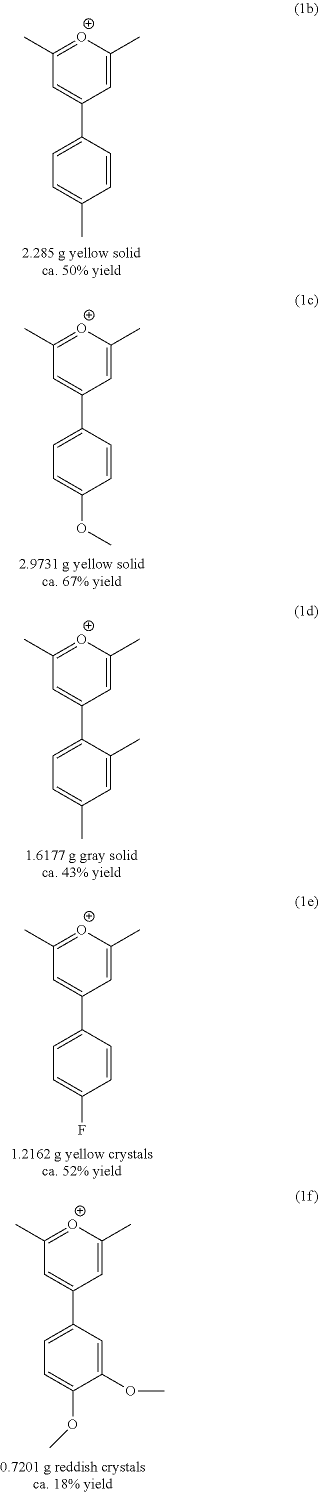

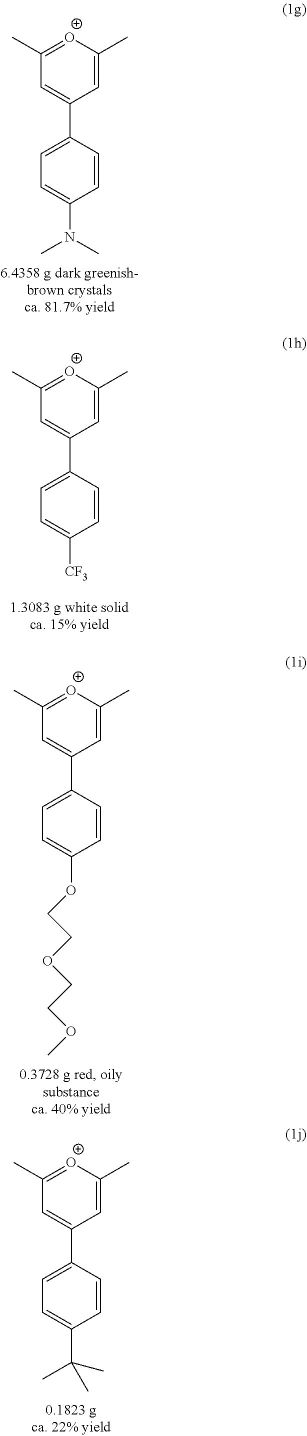

Alternatively, derivative (1a) could be synthesized following the procedure of Breit, et al. as shown in Reaction Scheme (D) below (Breit, B.; Winde, R.; Mackewitz, T.; Paciello, R.; Harms, K., Phosphabenzenes as Monodentate π-Acceptor Ligands for Rhodium-Catalyzed Hydroformylation. Chem. Eur. J. 2001, 7 (14), 3106-3121)) as follows:

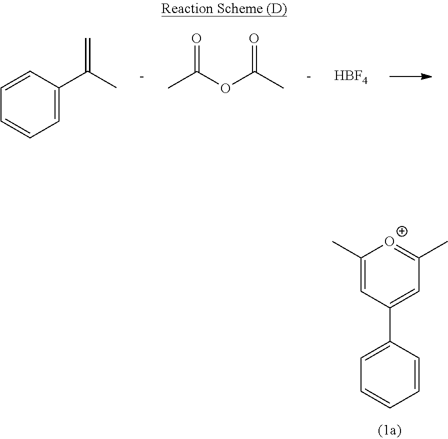

First, in a dry 25 mL round bottom flask equipped with a magnetic stir bar, acetic anhydride (3.5 milliliters, 37 millimoles) was mixed with tetrafluoroboric acid diethyl ether complex (0.572 milliliters, 4.2 millimoles). After cooling to zero degrees Celsius (0° C.) via ice bath, α-methylstyrene (0.546 milliliters, 4.2 millimoles) was added and the solution was stirred at zero degrees Celsius (0° C.) for 2 hours. After warming to room temperature overnight, the reaction mixture was poured over diethyl ether and the crude purple solid was isolated by filtration. Recrystallization from hot water afforded the product (1a) as yellow crystals (0.1215 g, 0.45 mmol) in 10% yield.

Pyrylium compounds (1a)-(1j) were prepared as BF₄⁻ salts, but conversion to other salts such as halides, hexafluorophophates, perchlorates, triflates, and bis(trifluoromethanesulfonyl)imides (triflimides), among others, can be achieved by simple anion metathesis. For example, the conversion of (1b) to the bis(trifluoromethanesulfonyl)imide salt was achieved by first charging a 2 L flask with 35 g of (1b)(BF₄) along with 600 ml of deionized water and 600 ml of methanol. 181 g of lithium bis(trifluoromethanesulfonyl)imide was added and the mixture was brought to a low boil, allowing the methanol to evaporate slowly over a few hours. Once most of the methanol had evaporated, heating was stopped, and the flask transferred to a refrigerator. After cooling overnight, the colorless/slightly yellow crystals were collected by vacuum filtration (isolated yield, 55.7 g, 95%).

2,6-dimethyl-1,4-diphenylpyridin-1-ium tetrafluoroborate ((2a) as shown below) was synthesized following the procedure of Huifeng, et al. as shown in Reaction Scheme (E) below (Huifeng, Y; Zhu, C.; Shen, L.; Geng, Q.; Hoch, K.; Yuan, T.; Cavallo, L.; Rueping, M., Nickel-catalyzed C—N bond activation: Activated primary amines as alkylating reagent in reductive cross-couplings. Chem. Sci. 2019, 10 (6), 4430-4435) as follows:

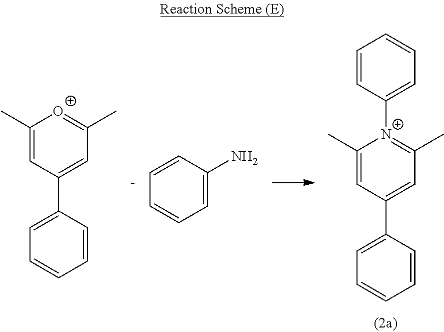

First, in a 50 milliliter round bottom flask equipped with a magnetic stir bar and condenser, 2,6-dimethyl-4-phenyl pyrylium tetrafluoroborate (0.3 grams, 1.1 millimole) was suspended in ethanol (20 mL). Aniline (0.12 milliliters, 1.3 millimoles) was added, and the mixture was refluxed at a refluxing temperature, here eighty eight degrees Celsius (88° C.), for 4 hours while under nitrogen. The solution was cooled to room temperature overnight and diluted with diethyl ether. The precipitate was isolated by filtration and dried under vacuum to afford the white solid (0.325 grams, 0.94 millimole) as the product according to formula (2a) above in 85% yield.

Other pyridinium compounds listed in Table 1 above were synthesized by following the same procedure with the appropriate amine or aniline reactant.

Embodiments for the disclosure can be described with reference to the following numbered clauses, with specific features laid out in the dependent clauses:

I. A pyridinium compound according to formula (1B):

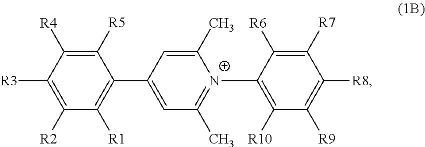

wherein:
R1 and R5 are independently selected from hydrogen, a methyl group, an ethyl group, a halide group, a halomethyl group, a perhalomethyl group, an alkoxy group, a nitrile group and a nitro group;
R2, R3 and R4 are independently selected from hydrogen, an unbranched alkyl group, a branched alkyl group, an alkoxy group, a haloalkyl group, an aralkyl group, an alkyl ether group, a nitro group, a nitrile group, a halide group, an acetyl group, a trialkylammonium alkyl group, an alkyl carboxylate group, an alkyl sulfonate group, and an alkyl phosphonate group;
R6 and R10 are independently selected from hydrogen, a methyl group, an ethyl group, a halide group, a halomethyl group, a perhalomethyl group, a hydroxyl group, an alkoxy group, a nitrile group and a nitro group;
R7 and R9 are independently selected from hydrogen, an unbranched alkyl group, a branched alkyl group, a haloalkyl group, a perhaloalkyl group, an aryl group, an aralkyl group, an alkyl ether group, a nitro group, a nitrile group, a halide group, an acetyl group, a trialkylammonium alkyl group, an alkyl carboxylate group, an alkyl sulfonate group, an alkyl phosphonate group, a tert-butyl group, a CF₃ group, —Br, an N,N-dimethylaniline group, and an alkyl triether group; and
R8 is independently selected from hydrogen, an unbranched alkyl, a branched alkyl, an alkoxy group, a haloalkyl group, a perhalomethyl group, an aryl group, an aralkyl group, an alkyl ether group, a nitro group, a nitrile group, a halide group, an acetyl group, a trialkylammonium alkyl group, an alkyl carboxylate group, an alkyl sulfonate group, an alkyl phosphonate group, a tert-butyl group, a CF₃ group, —Br, a N,N-dimethylaniline group, and an alkyl triether group,
wherein at least one of R1-R10 is not hydrogen.

II. The pyridinium compound according to clause I, wherein at least one of R1, R5, R6 or R10 is not hydrogen.

III. The pyridinium compound of clause I or clause II, wherein the pyridinium compound according to formula (1B) has a reduction potential from −1.54 to −1.83 V vs Fc/Fc⁺.

IV. The pyridinium compound according to any one of clauses I-III linked to an oxidizable moiety selected from a ferrocene, a carbazole, a phenazine, a phenoxazine, a phenothiazine, a phenothiazine-5-oxide, a phenothiazine-5,5-dioxide, a benzoquinone, and a cyclopropenium.

V. A pyridinium compound according to formula (1C):

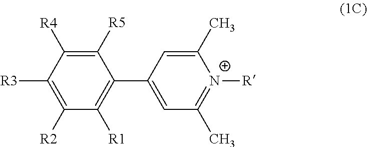

wherein:
R1 and R5 are independently selected from hydrogen, a methyl group, an ethyl group, a halide group, a halomethyl group, a hydroxyl group, an alkoxy group, a methoxy group, a nitrile group and a nitro group;
R2, R3 and R4 are independently selected from hydrogen, an unbranched alkyl group, a branched alkyl group, an alkoxy group, a methoxy group, a haloalkyl group, an aryl group, an aralkyl group, an alkyl ether group, a nitro group, a nitrile group, a halide group, an acetyl group, a trialkylammonium alkyl group, an alkyl carboxylate group, an alkyl sulfonate group, and an alkyl phosphonate group; and
R′ is selected from an unbranched alkyl group, a branched alkyl group, a haloalkyl group, an aralkyl group, an alkyl ether group, an alkyl nitro group, an alkyl nitrile group, a trialkylammonium alkyl group, an alkyl carboxylate group, an alkyl sulfonate group, and an alkyl phosphonate group,
wherein when at least one of R1, R2, R3, R4 and R5 is the methoxy group, R′ includes two carbons or more.

VI. The pyridinium compound according to clause V, wherein at least one of R1 or R5 is not hydrogen.

VII. The pyridinium compound of clause V or clause VI, wherein the pyridinium compound according to formula (1C) has a reduction potential from −1.54 to −1.83 V vs Fc/Fc⁺.

VIII. The pyridinium compound according to any one of clauses V-VII linked to an oxidizable moiety selected from a ferrocene, a carbazole, a phenazine, a phenoxazine, a phenothiazine, a phenothiazine-5-oxide, a phenothiazine-5,5-dioxide, a benzoquinone, and a cyclopropenium.

IX. A pyridinium compound selected from the group consisting of:

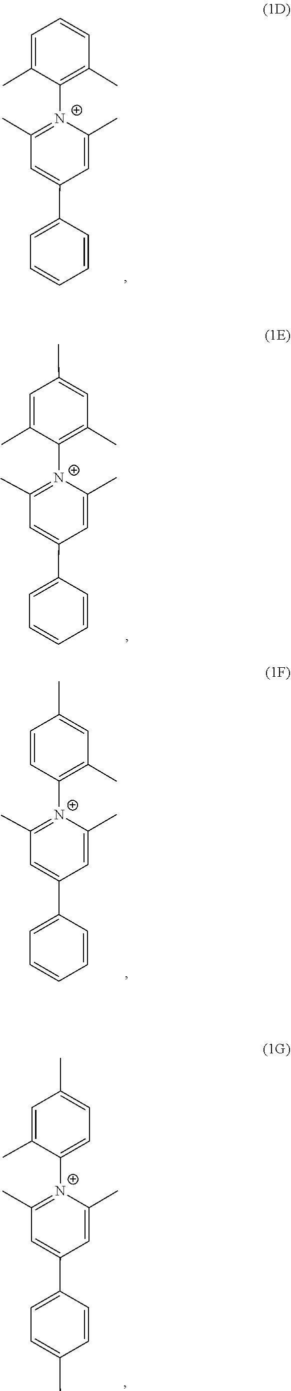

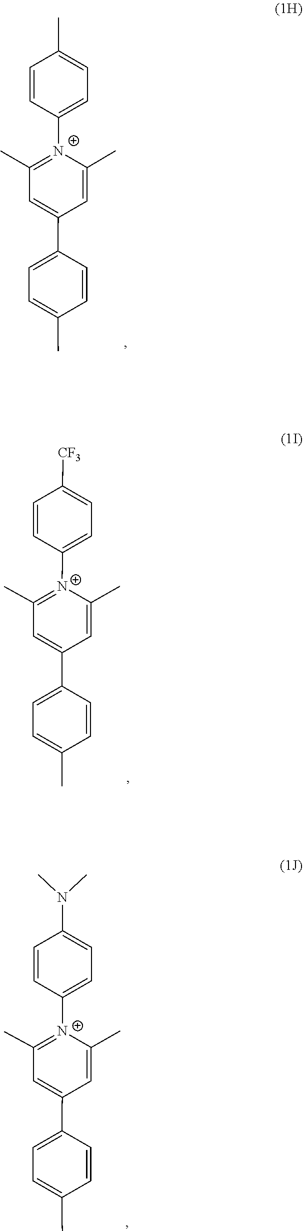

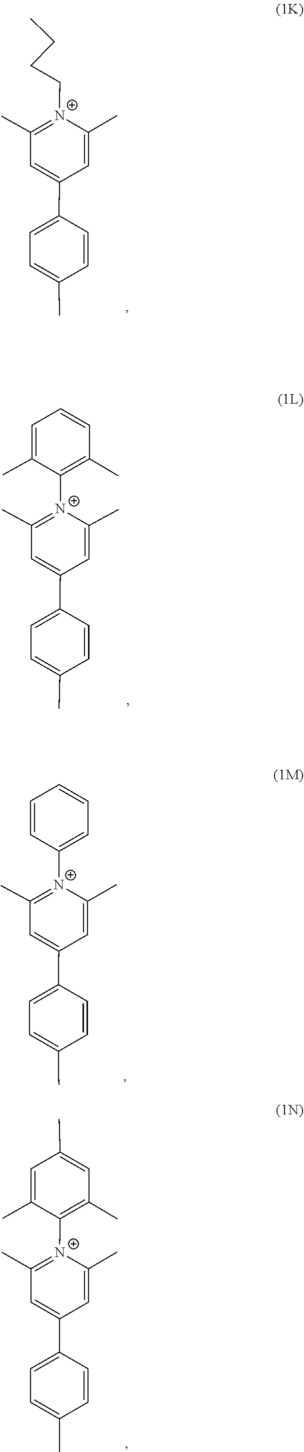

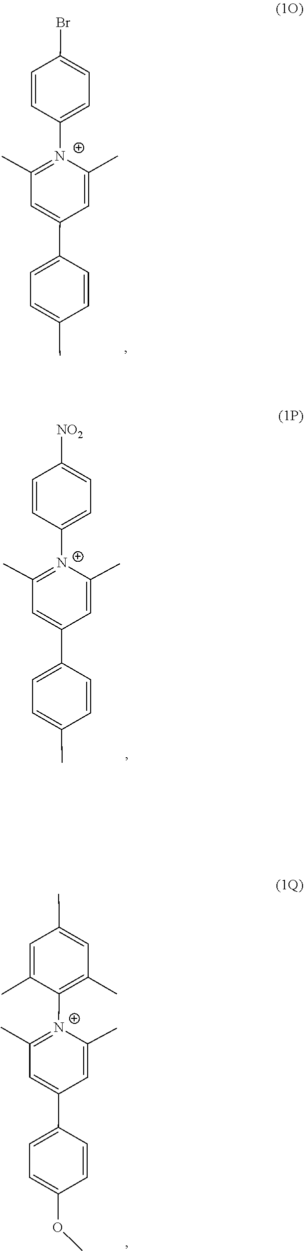

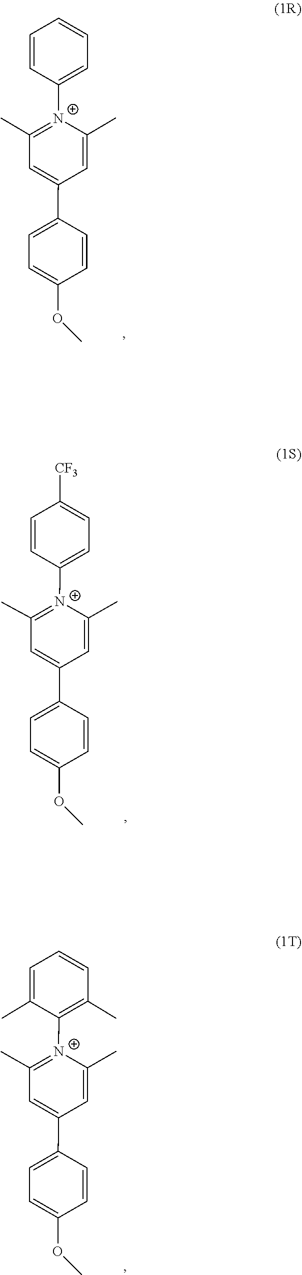

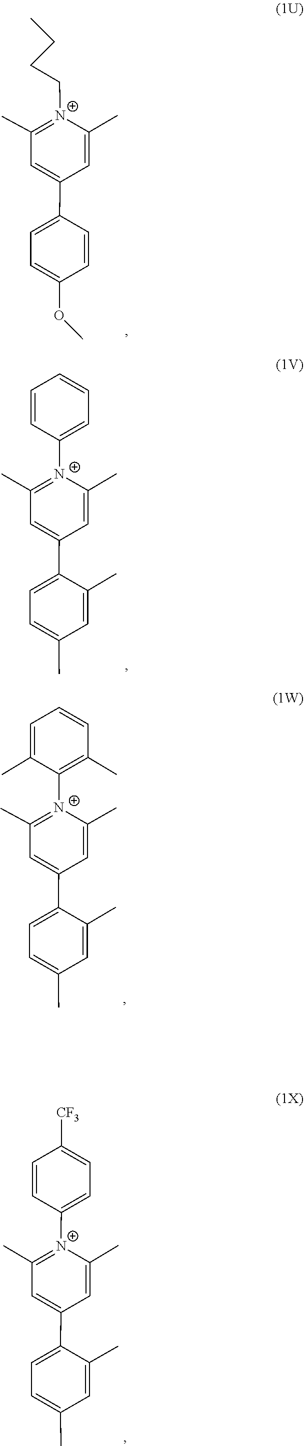

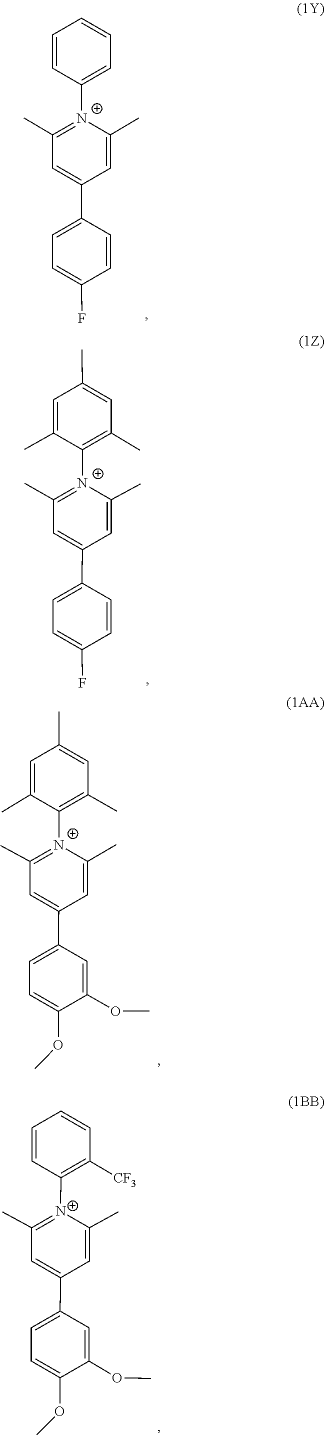

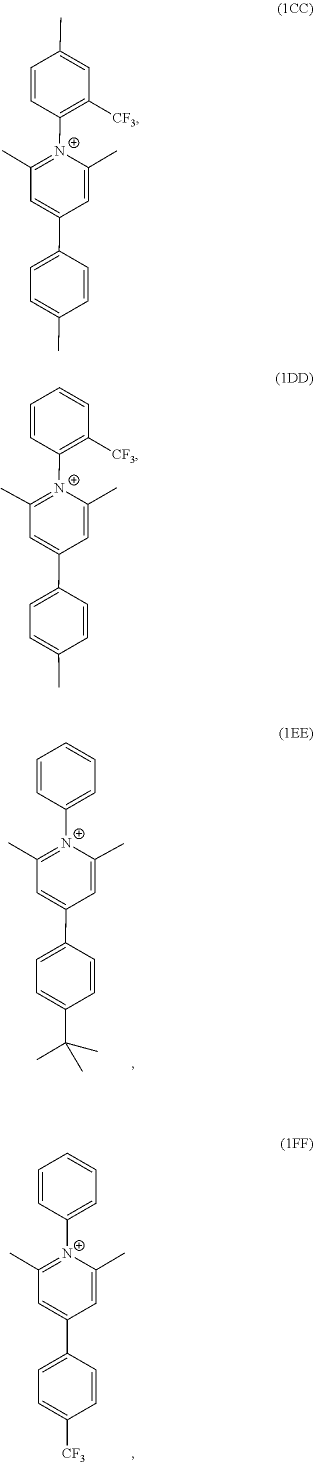

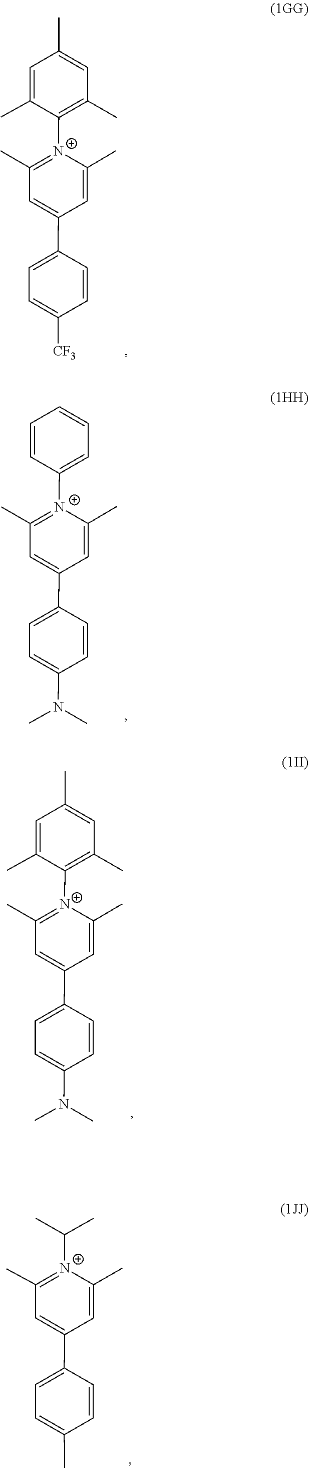

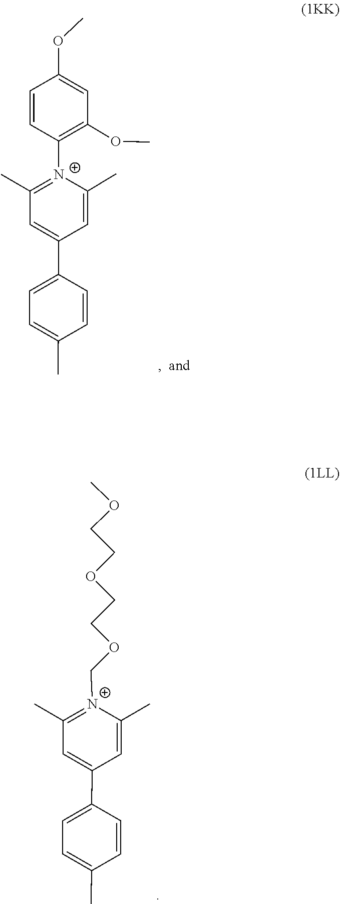

X. The pyridinium compound of clause IX, wherein the pyridinium compound according to formula (1C) has a reduction potential from −1.54 to −1.83 V vs Fc/Fc⁺.

XI. The pyridinium compound according to clause IX or clause X linked to an oxidizable moiety selected from a ferrocene, a carbazole, a phenazine, a phenoxazine, a phenothiazine, a phenothiazine-5-oxide, a phenothiazine-5,5-dioxide, a benzoquinone, and a cyclopropenium.

XII. A redox flow battery comprising:
(I) a cathode;
(II) an anode; and
(III) a charge-carrying electrolyte comprising the pyridinium compound according to any one of clauses I-IV.

XIII. A redox flow battery comprising:
(I) a cathode;
(II) an anode; and
(III) a charge-carrying electrolyte comprising the pyridinium compound according to any one of clauses V-VIII.

XIV. A redox flow battery comprising:
(I) a cathode;
(II) an anode; and
(III) a charge-carrying electrolyte comprising the pyridinium compound according to any one of clauses IX-XI.

Many modifications and variations of the subject application are possible in light of the above teachings. Therefore, the subject application may be practiced other than as specifically described.

1. A pyridinium compound according to formula (1B): 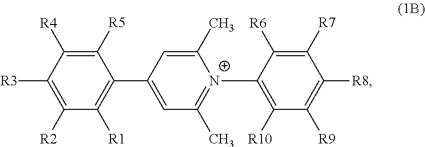
wherein:
R1 and R5 are independently selected from hydrogen, a methyl group, an ethyl group, a halide group, a halomethyl group, a perhalomethyl group, an alkoxy group, a nitrile group and a nitro group;
R2, R3 and R4 are independently selected from hydrogen, an unbranched alkyl group, a branched alkyl group, a haloalkyl group, an aralkyl group, an alkyl ether group, a nitro group, a nitrile group, a halide group, an acetyl group, a trialkylammonium alkyl group, an alkyl carboxylate group, an alkyl sulfonate group, and an alkyl phosphonate group;
R6 and R10 are independently selected from hydrogen, a methyl group, an ethyl group, a halide group, a halomethyl group, a perhalomethyl group, a hydroxyl group, an alkoxy group, a nitrile group and a nitro group;
R7 and R9 are independently selected from hydrogen, an unbranched alkyl group, a branched alkyl group, a haloalkyl group, a perhaloalkyl group, an aryl group, an aralkyl group, an alkyl ether group, a nitro group, a nitrile group, a halide group, an acetyl group, a trialkylammonium alkyl group, an alkyl carboxylate group, an alkyl sulfonate group, an alkyl phosphonate group, a tert-butyl group, a CF₃ group, —Br, an N,N-dimethylaniline group, and an alkyl triether group; and
R8 is independently selected from hydrogen, an unbranched alkyl, a branched alkyl, an alkoxy group, a haloalkyl group, a perhalomethyl group, an aryl group, an aralkyl group, an alkyl ether group, a nitro group, a nitrile group, a halide group, a trialkylammonium alkyl group, an alkyl carboxylate group, an alkyl sulfonate group, an alkyl phosphonate group, a tert-butyl group, a CF₃ group, —Br, a N,N-dimethylaniline group, and an alkyl triether group,
wherein at least one of R1-R10 is not hydrogen.

2. The pyridinium compound according to claim 1, wherein at least one of R1, R5, R6 or R10 is not hydrogen.

3. The pyridinium compound of claim 1, wherein the pyridinium compound according to formula (1B) has a reduction potential from −1.54 to −1.83 V vs Fc/Fc+.

4. The pyridinium compound according to claim 1 linked to an oxidizable moiety selected from a ferrocene, a carbazole, a phenazine, a phenoxazine, a phenothiazine, a phenothiazine-5-oxide, a phenothiazine-5,5-dioxide, a benzoquinone, and a cyclopropenium.

5. A pyridinium compound according to formula (1C): 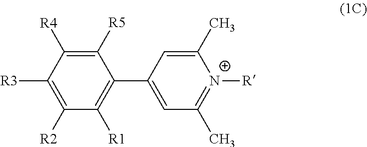
wherein:
R1 and R5 are independently selected from hydrogen, a methyl group, an ethyl group, a halide group, a halomethyl group, a hydroxyl group, an alkoxy group, a methoxy group, a nitrile group and a nitro group;
R2, R3 and R4 are independently selected from hydrogen, an unbranched alkyl group, a branched alkyl group, an alkoxy group, a methoxy group, a haloalkyl group, an aryl group, an aralkyl group, an alkyl ether group, a nitro group, a nitrile group, a halide group, an acetyl group, a trialkylammonium alkyl group, an alkyl carboxylate group, an alkyl sulfonate group, and an alkyl phosphonate group; and
R′ is selected from an unbranched alkyl group, a branched alkyl group, a haloalkyl group, an aralkyl group, an alkyl ether group, an alkyl nitro group, an alkyl nitrile group, a trialkylammonium alkyl group, an alkyl carboxylate group, an alkyl sulfonate group, and an alkyl phosphonate group,
wherein when at least one of R1, R2, R3, R4 and R5 is the methoxy group, R′ includes two carbons or more, and
wherein at least one of R1 or R5 is not hydrogen.

6. (canceled)

7. The pyridinium compound of claim 5, wherein the pyridinium compound according to formula (1C) has a reduction potential from −1.54 to −1.83 V vs Fc/Fc+.

8. The pyridinium compound according to claim 5 linked to an oxidizable moiety selected from a ferrocene, a carbazole, a phenazine, a phenoxazine, a phenothiazine, a phenothiazine-5-oxide, a phenothiazine-5,5-dioxide, a benzoquinone, and a cyclopropenium.

9. A pyridinium compound selected from the group consisting of:

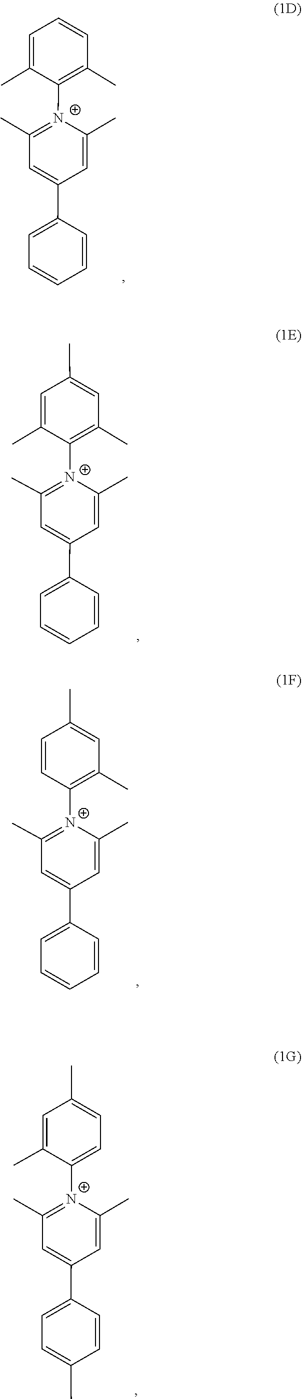

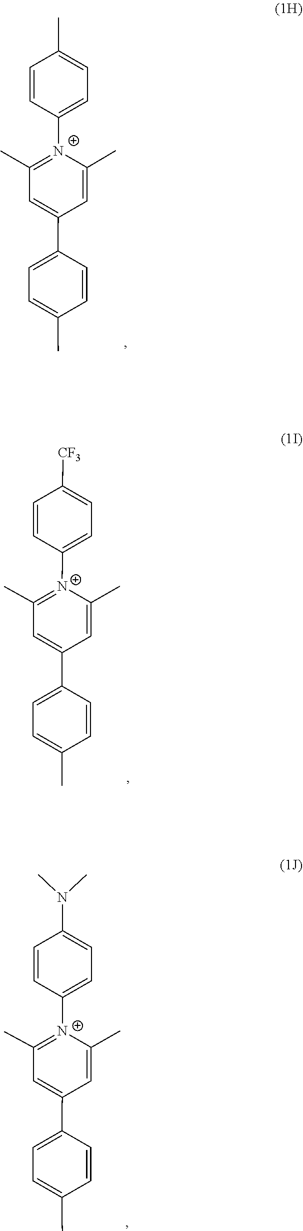

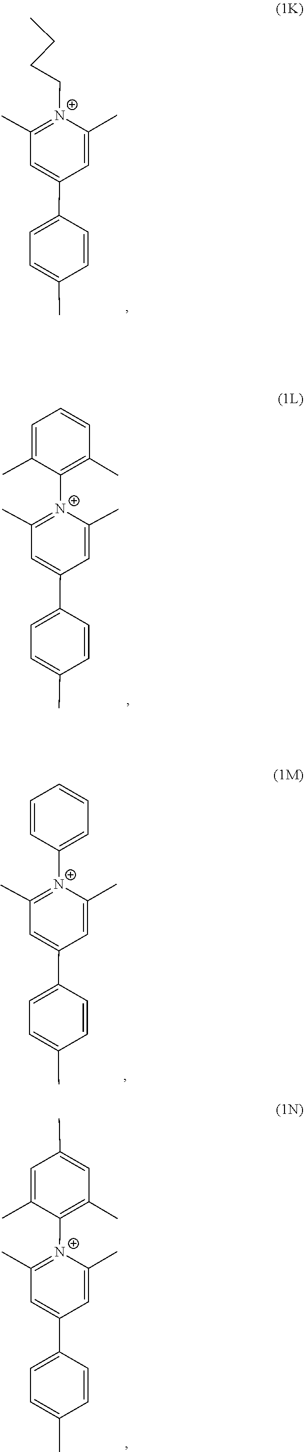

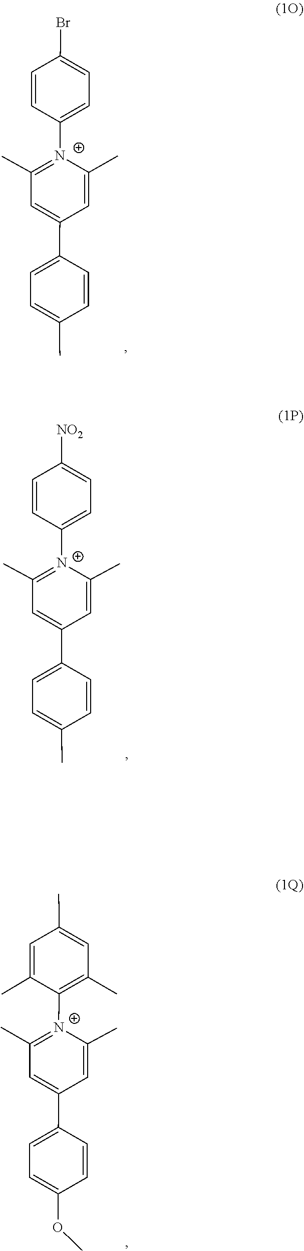

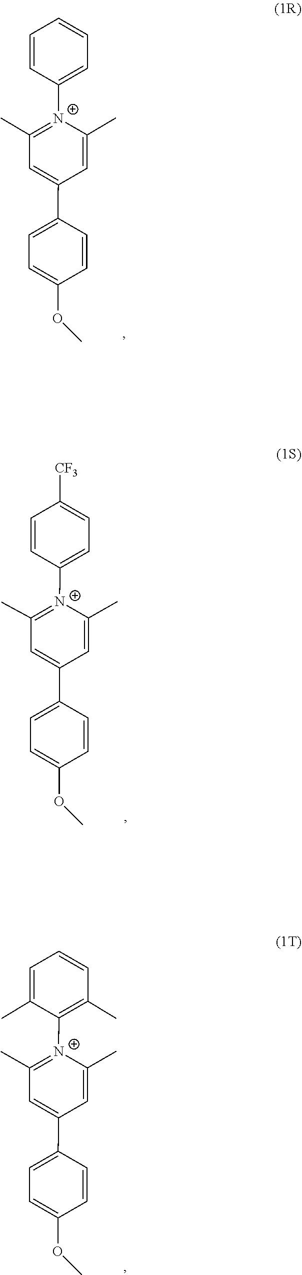

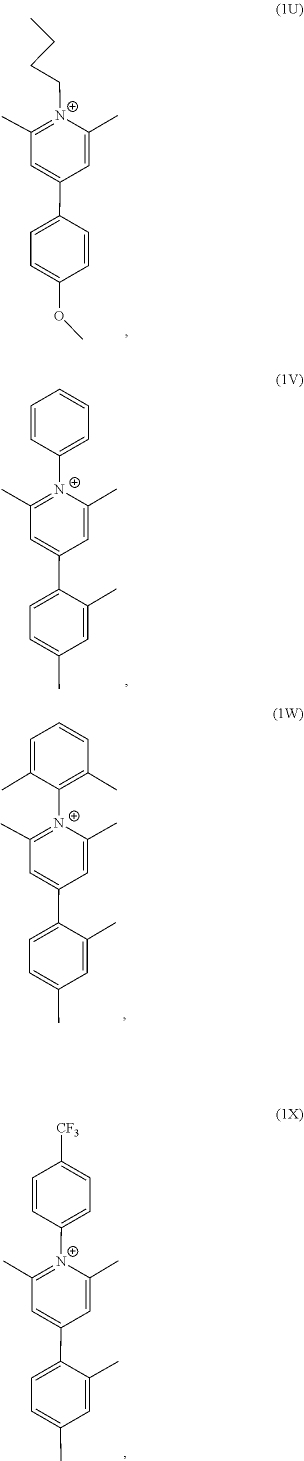

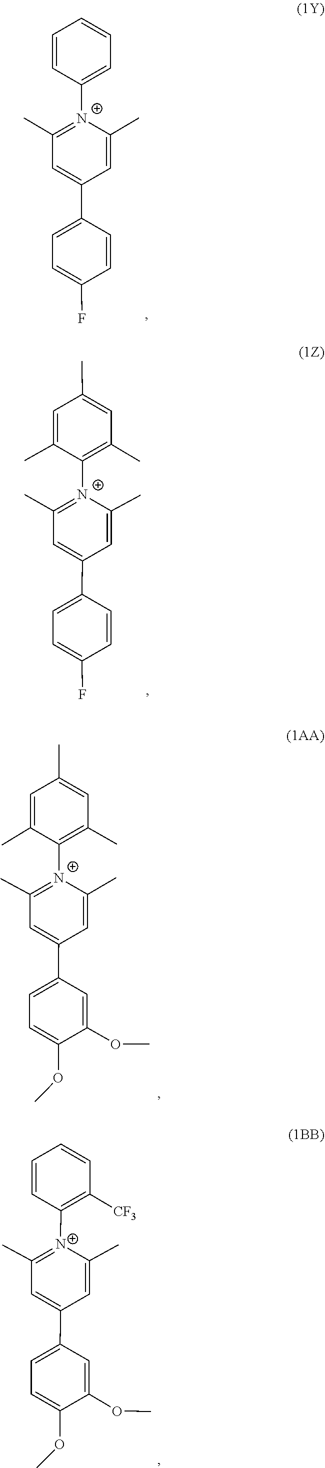

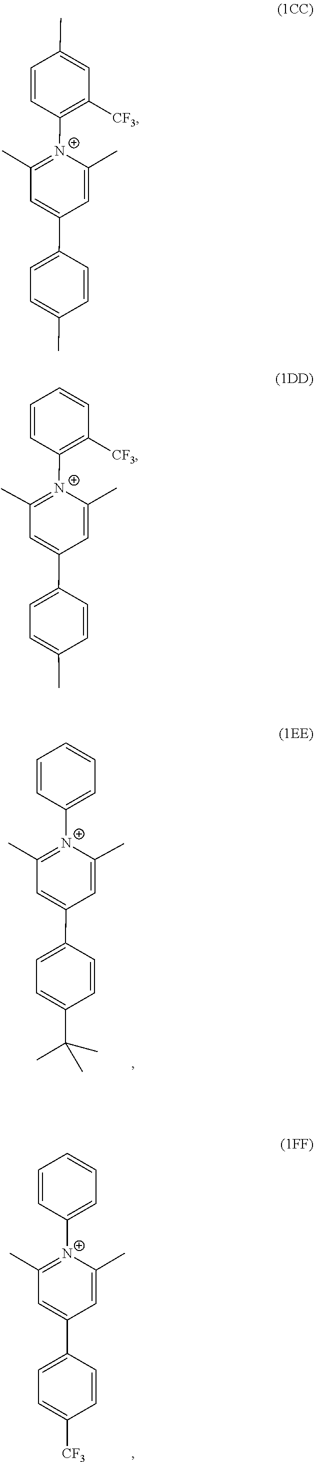

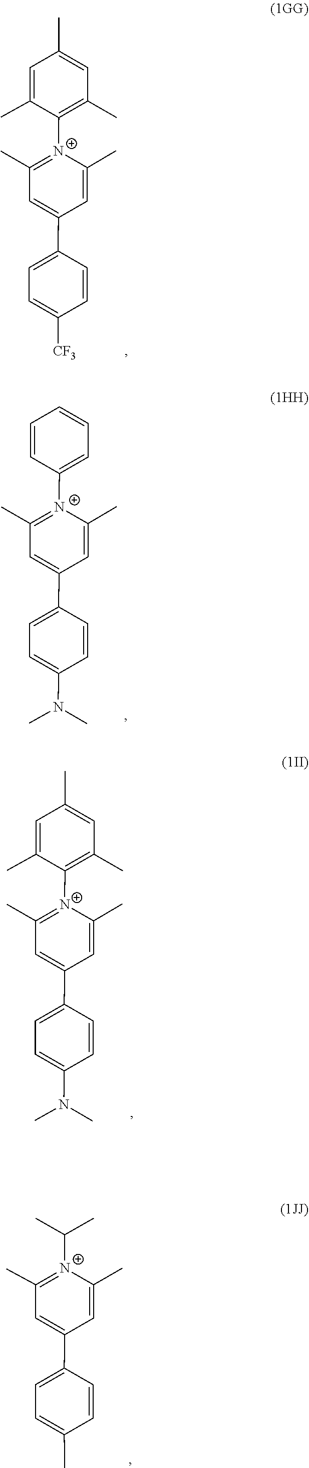

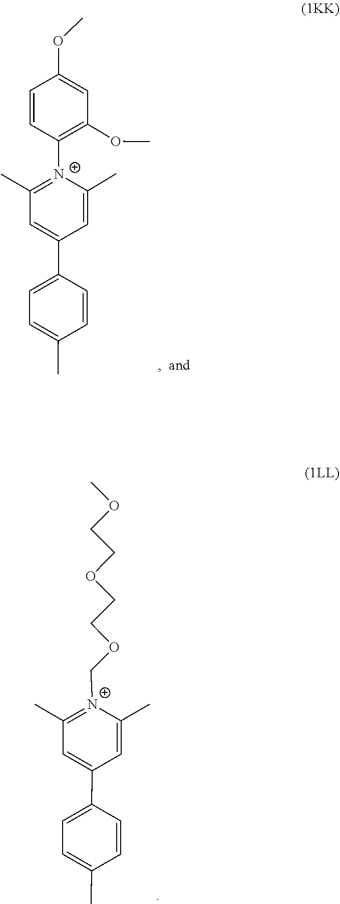

10. The pyridinium compound of claim 9, wherein the pyridinium compound according to formula (1C) has a reduction potential from −1.54 to −1.83 V vs Fc/Fc+.

11. The pyridinium compound according to claim 9 linked to an oxidizable moiety selected from a ferrocene, a carbazole, a phenazine, a phenoxazine, a phenothiazine, a phenothiazine-5-oxide, a phenothiazine-5,5-dioxide, a benzoquinone, and a cyclopropenium.

12. A redox flow battery comprising:
(I) a cathode;
(II) an anode; and
(III) a charge-carrying electrolyte comprising the pyridinium compound according to claim 1.

13. A redox flow battery comprising:
(I) a cathode;
(II) an anode; and
(III) a charge-carrying electrolyte comprising the pyridinium compound according to claim 5.

14. A redox flow battery comprising:
(I) a cathode;
(II) an anode; and
(III) a charge-carrying electrolyte comprising the pyridinium compound according to claim 9.
